(19) United States
(12) Patent Application Publication
(10) Pub. No.: US 2025/0391542 A1
(43) Pub. Date: Dec. 25, 2025

(54) SYSTEMS AND METHODS FOR DIFFERENTIATING BETWEEN TISSUES DURING SURGERY

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Viviane Tabar, New York, NY (US); Rabih Bou Nassif, New York, NY (US)

(21) Appl. No.: 18/877,158

(22) PCT Filed: Jun. 21, 2023
(86) PCT No.: PCT/US2023/068826

§ 371 Date: Dec. 19, 2024

Related U.S. Application Data
(60) Provisional application No. 63/354,859, filed on Jun. 23, 2022.
(60) Provisional application No. 63/487,502, filed on Feb. 28, 2023.

(51) Int. Cl. G16H 30/40; G06T 7/00; G16H 20/40; G16H 30/20; G16H 50/20

(52) U.S. Cl. CPC G16H 30/40; G06T 7/0012; G16H 20/40; G16H 30/20; G16H 50/20; G06T 2207/20081; G06T 2207/20084

(57) ABSTRACT

Presented herein are systems and methods relating to artificial intelligence-drive intraoperative diagnosis. For example, a method can include capturing, by an optical reader device of a mobile device, an image of a tissue. A method can further include providing, by a mobile application of the mobile device, the image of the tissue to a tissue analysis circuit. A method can include receiving, from the tissue analysis circuit via the mobile device, a tissue classification. A method can include presenting, via a graphical user interface of the mobile device, a display screen comprising the tissue classification.

### CROSS REFERENCE TO RELATED APPLICATION

This application claims the benefit of and priority to U.S. Provisional Patent Application No. 63/354,859, entitled “SYSTEMS AND METHODS FOR DIFFERENTIATING BETWEEN TISSUES DURING SURGERY,” filed Jun. 23, 2022, and U.S. Provisional Patent Application No. 63/487,502, entitled “SYSTEMS AND METHODS FOR DIFFERENTIATING BETWEEN TISSUES DURING SURGERY,” filed Feb. 28, 2023, the entireties of which are incorporated by reference herein.

### BACKGROUND

A computing device may employ computer vision techniques to compare different images to one another. In comparing the images, the computing device may use any number of factors to perform the evaluation.

### SUMMARY

At least one aspect of the present disclosure is directed to a method. The method can include capturing, by an optical reader device of a mobile device, an image of a tissue. A method can further include providing, by a mobile application of the mobile device, the image of the tissue to a tissue analysis circuit. A method can include receiving, from the tissue analysis circuit via the mobile device, a tissue classification. A method can include presenting, via a graphical user interface of the mobile device, a display screen comprising the tissue classification.

In some implementations, the method can include processing, by the mobile application, the image of the tissue prior to providing the image of the tissue to the tissue analysis circuit. Processing the image of the tissue can include at least one of resizing the image, reformatting the image, or applying a filter to the image.

In some implementations, the method can include the display screen further including the image of the tissue, wherein the tissue classification comprises a pop-up window within the first display screen.

In some implementations, the method can include the display screen presented via the graphical user interface less than one minute after the image of the tissue is provided to the tissue analysis circuit.

In some implementations, the method can include determining, by the mobile application, that the image of the tissue needs to be reformatted according to a tissue analysis specification. The method can include reformatting, by the mobile application prior to providing the image of the tissue to the tissue analysis circuit, the image of the tissue according to the tissue classification in response to the determination that the image of the tissue needs to be reformatted.

In some implementations, the method can include the mobile application including the tissue analysis circuit.

In some implementations, the method can include receiving, from the tissue analysis circuit via the mobile application, a request for a second image of the tissue. The method can include presenting, via the graphical user interface of the mobile device, a second display screen comprising the request for the second image of the tissue.

In some implementations, the method can include the tissue classification based on an automated neural network analysis performed by a neural network. The neural network analysis can compare the image of the tissue with a dataset.

In some implementations the method can include the dataset including a normal tissue image dataset and an abnormal tissue image dataset. The neural network can be a pretrained neural network that is trained to classify the image of the tissue as normal or abnormal.

In some implementations, the method can include the image of the tissue including at least a portion of a generated tissue image, the generated tissue image comprising a Stimulated Raman Histology (SRH) image.

At least one aspect of the present disclosure is directed to an apparatus. The apparatus can be a mobile device. The mobile device can include a processing circuit having a processor and a memory. The memory can store instructions that, when executed by the processor, cause the processor to receive an image of a tissue. The instructions that, when executed by the processor can cause the processor to provide the image of the tissue to a tissue classification circuit. The instructions that, when executed by the processor can cause the processor to receive, by the tissue classification circuit based on an automated neural network analysis, a classification of the image of the tissue. The instructions that, when executed by the processor can cause the processor to present, via a display device, a display screen comprising the classification of the image of the tissue, the classification comprising an indication that the tissue is normal or abnormal.

In some implementations, the mobile device can include an optical reader configured to capture an image. The optical reader can capture image of the tissue from a generated Stimulated Raman Histology image displayed on an imaging device.

In some implementations, the mobile device can include the image classification circuit including a neural network. The neural network can perform the automated neural network analysis. The neural network can be trained to classify the image of the tissue as normal or abnormal using a normal tissue image dataset and an abnormal tissue dataset.

In some implementations, the mobile device of claim can include the instructions to further cause the processor to process, by the mobile device, the image of the tissue prior to providing the image of the tissue to the tissue analysis circuit. Processing the image of the tissue can include at least one of resizing the image, reformatting the image, or applying a filter to the image.

In some implementations, the mobile device can include the instructions to further cause the processor to determine, by the mobile device, that the image of the tissue needs to be reformatted according to a tissue analysis specification. The instructions can further cause the processor to reformat, by the mobile device prior to providing the image of the tissue to the tissue analysis circuit, the image of the tissue according to the tissue classification in response to the determination that the image of the tissue needs to be reformatted.

In some implementations, the mobile device can include the first display screen presented via the display device less than one minute after the image of the tissue is provided to the tissue analysis circuit.

At least one aspect of the present invention is directed to a system. The system can include an imaging device. The imaging device can include a display device. The imaging device can generate a Stimulated Raman Histology (SRH) image of a tissue and display the image on the display device. The system can include a tissue classification computer system coupled to the imaging device. The tissue classification computer system can include a neural network trained with a normal tissue image dataset and an abnormal tissue image dataset. The tissue classification computer system can receive the SRH image of the tissue. The tissue classification computer system can perform an automated neural network analysis to classify at least a portion of the SRH image of the tissue as normal or abnormal. The tissue classification computer system can provide an indication of a classification of the SRH image of the tissue as normal or abnormal.

In some implementations, the system can include the neural network, where the neural network is a pre-trained neural network that is trained using a normal tissue image dataset and an abnormal tissue image dataset to classify an image of tissue as normal or abnormal.

In some implementations, the system can include the tissue classification computer system to select a portion of the SRH image of the tissue, wherein the automated neural network analysis is performed on the selected portion of the SRH image of the tissue.

In some implementations, the system can include the indication of the classification of the SRH image of the tissue provided by the image classification computer system to the display device of the imaging device.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts a block diagram of a mobile device, according to an embodiment.

FIG. 2 depicts a block diagram of a mobile device including a neural network, according to an embodiment.

FIG. 3 depicts a system for classifying an image of tissue, according to an embodiment.

FIG. 4 depicts a flow diagram of a method for classifying an image of a tissue, according to an embodiment.

FIG. 5 depicts a block diagram of a server system and a client computer system in accordance with an illustrative embodiment.

FIG. 6 depicts a flow diagram of developing and deploying a mobile application for classifying an image of a tissue, according to an embodiment.

FIG. 7 depicts a distribution of a certainty score for a mobile application for classifying an image of a tissue, according to an embodiment.

FIG. 8 depicts a distribution of a certainty score for a mobile application for classifying an image of a tissue, according to an embodiment.

FIG. 9 depicts a distribution of a certainty score for a mobile application for classifying an image of a tissue, according to an embodiment.

### DETAILED DESCRIPTION

Following below are more detailed descriptions of various concepts related to, and embodiments of, systems and methods for artificial intelligence-drive intraoperative diagnosis. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes systems and methods for differentiating between tissues during surgery.

Section B describes systems and methods for using images to train a deep learning model for differentiating between different tissues during surgery.

Section C describes a network environment and computing environment which may be useful for practicing various embodiments described herein.

### A. Systems and Methods for Differentiating Between Tissues During Surgery

Accurate classification of a tissue sample during a surgery is important to ensure that the appropriate tissues (e.g., tumorous, cancerous, etc.) are excised, while other tissues (e.g., normal, healthy) are not inadvertently excised. Accordingly, it is necessary for a tissue to be analyzed to determine whether the tissue is normal or abnormal. In conventional practice, a tissue specimen is removed from a patient during surgery and is then examined by a pathologist who determines that the tissue is normal or abnormal. Based on the pathologist's determination, a surgeon may proceed to excise certain tissue from a patient. The pathologist typically operates from a pathology lab or department of a hospital, which can be located away from an operating room where a patient's surgery occurs. Moreover, the pathologist may not be present in the pathology lab or pathology department during a time period when a surgeon needs a tissue specimen analyzed (i.e., when the patient is in surgery). A pathologist's analysis of tissue specimen can take 30 to 50 minutes, in some examples. Any delay in analyzing a tissue specimen undesirably exposes a patient to increased risk. In some instances, a specialized pathologist may not be available, such as in developing countries or isolated geographic locations, for example.

The systems and methods disclosed herein are partially focused on tissue imaging and pathology (anatomic pathology, histopathology, cytopathology, dermatopathology, chemical pathology, immunopathology, hematology/hematopathology, cytology, molecular analysis) with tissue and cell analysis (nuclear/chromatin profile, cell density, cell density scores, biochemical cell information, statistical analysis). For example, the analysis of cell density or cell density scores can provide for the quantification of tumor invasion of a tissue. Tissue imaging and pathology can use both classic and innovative data collection and imaging techniques. However, the systems and methods disclosed herein can also be used in other context unrelated to tissue analysis, for example.

In some scenarios, a Raman Spectroscopy Imaging device can be used in an operating room to generate an image of a tissue sample. For example, a Raman Spectroscopy device can be used to generate a Stimulated Raman Histology (SRH) image of a tissue sample. Though the discussion that follows references SRH images, it is understood that images from other devices, such as magnetic resonance imaging (MRI) machine, a computed tomography (CT) scan device, a computerized axial tomography (CAT) scan device, an ultrasound imaging device, an X-Ray imaging device, or other imaging device, can be used to generate an image of a tissue sample, for example. The SRH image can be displayed on a display device of the Raman Spectroscopy device to provide an accurate image of a tissue that includes optical and chemical information. In one example, the SRH image can comprise a biochemical “fingerprint” of the tissue sample by providing information regarding multiple biological molecules of the tissue in the form of an image. The SRH image can be generated in a short period of time (e.g., three minutes, five minutes, one minute, etc.).

According to the present disclosure, a tissue differentiation system can be used to analyze the SRH image in order to determine a characteristic about the tissue. For example, the system can analyze the tissue depicted in the SRH image to determine if the tissue is normal (e.g., healthy) or abnormal (e.g., tumorous, cancerous, etc.). The tissue differentiation system can include a mobile device (e.g., a cellular phone, a tablet computer, a laptop computer, etc.). The mobile device can include a tissue analysis circuit comprising a neural network. The tissue analysis circuit and the neural network can analyze an image of a tissue and can generate a tissue classification that classifies the tissue as normal or abnormal. For example, an image of tissue proximate to an edge or margin of a tumor can be analyzed to determine whether a portion of the image of the tissue is tumorous or non-tumorous. The image of the tissue can be captured by an optical device (e.g., a camera or webcam of the mobile device) from an SRH image displayed on a display device of the Raman Spectroscopy device. The tissue analysis circuit and the neural network can present a tissue classification via a graphical use interface of the mobile device within one minute after the image of the tissue is captured. Accordingly, the system can provide a surgeon or medical professional with an indication that the tissue is normal or abnormal within a short period of time without requiring time-consuming analysis by a pathologist, thereby reducing the risk to a patient.

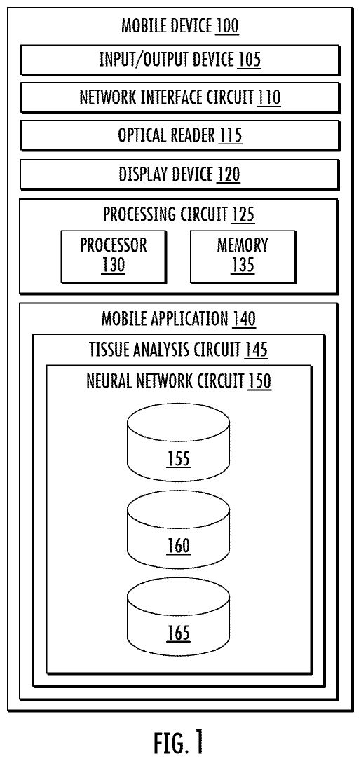

Referring now to FIG. 1, a mobile device 100 is shown. The mobile device 100 can include an input/output device 105, a network interface circuit 110, an optical device 115, a display device 120, a processing circuit 125, and a mobile application 140. The processing circuit 125 can include a processor 130 and a memory 135. The mobile application 140 can include a tissue analysis circuit 145. The tissue analysis circuit 145 can include or be coupled with a neural network circuit 150. In one example, the mobile device 100 can analyze an image of tissue to determine if the tissue is normal or abnormal. In another example, the mobile device 100 can distinguish one tissue from another tissue. In yet another embodiment, the mobile device 100 can be used to determine a characteristic of a tissue during a surgical operation (e.g., a tumor removal procedure).

The mobile device 100 may be used by a user, such as a surgeon, nurse, pathologist, medical technician, or other medical professional. In one example, the user can use the mobile device 100 to perform various actions, such as capturing an image of a tissue, providing the captured image of the tissue to a tissue analysis circuit, receiving a tissue classification regarding the image of the tissue, and providing, via a graphical user interface, a tissue classification to the user. The mobile device 100 is structured to exchange data over at least one wireless network via the network interface circuit 110, execute software applications, access websites, generate graphical user interfaces, and perform other operations that are typical of mobile devices or at least as described herein. The mobile device 100 may be, for example, a cellular phone, smart phone, mobile handheld wireless e-mail device, personal digital assistant, portable gaming device, a tablet computing device, or other suitable device.

The input/output device 105 of the mobile device 100 can include hardware and associated logic (e.g., instructions, computer code, etc.) to enable the mobile device 100 to exchange information with a user and other devices (e.g., a remotely-located computing system) that may interact with the mobile device 100. The input/output device 105 can be an input-only device (e.g., a button), an output-only device, or be a combination input/output devices. The input aspect of the input/output device 105 allows the user to input or provide information into the mobile device 100, and may include, for example, a mechanical keyboard, a touchscreen, a microphone, a camera (e.g., optical device 115), a fingerprint scanner, a device engageable to the mobile device 100 via a connection (e.g., USB, serial cable, Ethernet cable, etc.), and so on. The output aspect of the input/output device 105 allows the user to receive information from the mobile device 100, and may include, for example, a digital display, a speaker, illuminating icons, light emitting diodes (“LEDs”), and so on. For example, the input/output device 105 can provide results of a tissue analysis or other analysis via text (e.g., by the display device) or via some other notification (e.g., a speaker, a text message transmitted to a mobile phone, etc.). The input/output device 105 may also include systems, components, devices, and apparatuses that serve both input and output functions. Such systems, components, devices and apparatuses may include, for example, radio frequency (“RF”) transceivers, near-field communication (“NFC”) transceivers, and other short range wireless transceivers (e.g., Bluetooth®, laser-based data transmitters, etc.). The input/output device 105 may also include other hardware, software, and firmware components that may otherwise be needed for the functioning of the mobile device 100.

The network interface circuit 110 can include one or more antennas or transceivers and associated communications hardware and logic (e.g., computer code, instructions, etc.). The network interface circuit 110 is structured to allow the mobile device 100 to access and couple/connect to a wireless network to, in turn, exchange information with another device (e.g., a remotely-located computing system). The network interface circuit 110 allows for the mobile device 100 to transmit and receive internet data and telecommunication data. Accordingly, the network interface circuit 110 includes any one or more of a cellular transceiver (e.g., CDMA, GSM, LTE, etc.), a wireless network transceiver (e.g., 802.11X, ZigBee®, WI-FI®, Internet, etc.), and a combination thereof (e.g., both a cellular transceiver). Thus, the network interface circuit 110 enables connectivity to WAN as well as LAN (e.g., Bluetooth®, NFC, etc. transceivers). Further, in some embodiments, the network interface circuit 110 includes cryptography capabilities to establish a secure or relatively secure communication session between other systems such as a remotely-located computer system, a second mobile device associated with the user or a second user, the a patient's computing device, and/or any third-party computing system. In this regard, information (e.g., confidential patient information, images of tissue, results from tissue analyses, etc.) may be encrypted and transmitted to prevent or substantially prevent a threat of hacking or other security breach.

The optical device 115 can be a camera that can record or capture still images, moving images, time lapse images, etc. For example, the optical device 115 could be an integrated camera of the mobile device 100 (e.g., a cell phone camera) than can be front-facing, rear-facing etc. relative to the display device 120 of the mobile device 100. The optical device 115 can also be a separate camera device (e.g., a web cam, portable camera, borescope, etc.) that can be in communication with the mobile device. For example, the optical device could be a portable camera that communicates wirelessly with the mobile device 100 via the network interface circuit 110 to provide image data to the mobile device. In some examples, the mobile device 100 can include a plurality of optical devices 115.

The display device 120 can be or include an LCD screen, LED screen, touch screen, or similar device. For example, the display device 120 can be a touch screen of the mobile device 100 that is configured to display or present an image or graphical user interface to the user. The mobile device 100 may generate and/or receive and present various display screens on the display device 120. For example, a graphical user interface relating to classification of a tissue sample (e.g., a tissue classification widget) may be generated by the mobile device 100 presented to the user via the display device 120. In other examples, the user may interact with the mobile device 100 via the display device 120. For example, the user can provide an input to the mobile device 100 by touching (e.g., taping, dragging, etc.) the display device 120 with a finger, stylus, or other object. In another example, the mobile device 100 can include a plurality of display devices 120 that can be configured to display or present information to the user.

The processing circuit 125 can include the processor 130 and the memory 135. The processing circuit 125 can be communicably coupled with the mobile application 140, the tissue analysis circuit 145, or the neural network circuit 150. For example, the mobile application 140, the tissue analysis circuit 145, and/or the neural network circuit 150 can be executed by the processor 130 of the processing circuit 125. The processor 130 can be coupled with the memory 135. The processor 130 can be a general purpose or specific purpose processor, an application specific integrated circuit (ASIC), one or more field programmable gate arrays (FPGAs), a group of processing components, or other suitable processing components. The processor 130 is configured to execute computer code or instructions stored in the memory 135 or received from other computer readable media (e.g., CDROM, network storage, a remote server, etc.).

The memory 135 can include one or more devices (e.g., memory units, memory devices, storage devices, etc.) for storing data and/or computer code for completing and/or facilitating the various processes described in the present disclosure. The memory 135 may include random access memory (RAM), read-only memory (ROM), hard drive storage, temporary storage, non-volatile memory, flash memory, optical memory, or any other suitable memory for storing software objects and/or computer instructions. The memory 135 may include database components, object code components, script components, or any other type of information structure for supporting the various activities and information structures described in the present disclosure. The memory 135 may be communicably connected to the processor 130 via processing circuit 125 and may include computer code for executing (e.g., by the processor 130) one or more of the processes described herein. For example, the memory can include or be communicably coupled with the processor 130 to execute instructions related to the mobile application 140, the tissue analysis circuit 145, or the neural network circuit 150. In one example, the memory 135 can include or be communicably coupled with the mobile application 140, the tissue analysis circuit 145, or the neural network circuit 150. In one example, the mobile application 140, the tissue analysis circuit 145, or the neural network circuit 150 can be stored on a separate memory device located remotely from the mobile device 100 that is accessible by the processing circuit 125 via the network interface circuit 110.

The mobile application 140 can be a mobile application 140 operated on the mobile device 100 that allows a user to perform various operations related to analyzing a tissue sample. For example, the mobile application 140 can be structured to facilitate a user's analysis of an image of a tissue sample (e.g., an SRH image produced by a Raman Spectroscopy machine, an MRI machine, a CT device, or other imaging device) to determine whether the tissue depicted in the image is normal tissue or abnormal tissue. For example, the mobile application 140 can allow the user to capture or upload an image of a tissue sample to be analyzed via a graphical user interface presented on the display device 120. In another example, the mobile application 140 can facilitate an analysis of the image of the tissue sample. In yet another example, the mobile application 140 can present a tissue classification result to the user, such as by providing a notification via a graphical user interface presented on the display device 120. In various examples, the mobile application 140 can allow a user to provide an image of a tissue for analysis and subsequently present the user with a classification of the tissue depicted in the image, where the classification can be presented within a short period of time after the image is provided for analysis (e.g., less than three minutes, approximately one minute, etc.).

The mobile application 140 can be configured to receive an image as an input. For example, the mobile application 140 can be communicably coupled with the optical device 115 and can receive an image captured by the optical device 115. In one example, the mobile application 140 can obtain, from a photo library or image database of the memory 135 of the mobile device 100, a previously-captured image of a tissue sample. In another example, the mobile application 140 can receive an image of a tissue sample immediately upon capture of the image by the optical device 115. In yet another example, the mobile application 140 can include a camera function (e.g., camera application) that allows the mobile application 140 to control the optical device 115 to capture an image of a tissue sample. The mobile application 140 can be configured to alter an image of a tissue sample to prepare it for analysis or for some other purpose. For example, the mobile application 140 can reformat an image of a tissue sample to ensure the image has proper dimensions (e.g., 224 pixels by 224 pixels, etc.) or has the proper file size (e.g., 1 Mb, less than 1 Mb, less than 5 Mb, less than 200 Mb, etc.). In another example, the mobile application 140 can ensure that the color of the image is properly calibrated or expressed by converting the image to be compatible with RGB (Red, Green, Blue) color code. The mobile application 140 can crop, rotate, invert, or resize the image of a tissue sample, according to some examples.

The mobile application 140 can receive a user input regarding the image of the tissue sample. For example, the mobile application 140 can receive data (e.g., information, a command, etc.) regarding the tissue sample via a user input provided via the display device 120 or an input/output device 105. The data regarding the tissue sample can relate to, for example, a tissue sample location on a patient (e.g., abdominal tissue, pituitary tissue, etc.), demographic information about the patient, or otherwise. In some examples, the data regarding the tissue sample can inform a subsequent tissue analysis by ensuring that a tissue analysis function is properly calibrated or is analyzing the image of the tissue sample with reference to an appropriate sample of known tissue images. In one example, the mobile application 140 can reformat or modify the image of the tissue sample based on the data regarding the tissue sample. For example, the mobile application 140 can resize the image to a particular size that is associated with the particular type of tissue specified by the data regarding the tissue sample. The data regarding the tissue sample can be embedded in the image of the tissue sample or otherwise associated with the image of the tissue sample.

The mobile application 140 can receive information from another computing system related to the patient, the tissue sample, the medical procedure being performed on the patient, or otherwise. For example, the mobile application 140 can communicate with a hospital or medical center computer system to retrieve medical records related to the patient or to receive other pertinent information regarding the patient, the associated medical professionals, the medical procedure, or otherwise. The mobile application 140 can wirelessly communicate with the hospital computer system using end-to-end encryption techniques, according to one example. The mobile application 140 may provide information to another computing system. For example, the mobile application 140 can provide the image of the tissue sample or patient information to a hospital computing system. The image of the tissue sample or the patient information can be stored in a database of the hospital computing system. The mobile application 140 can prompt the hospital computing system to create a new entry in a patient database, for example.

The mobile application 140 can pre-analyze the image of a tissue sample prior to providing the image of the tissue sample for tissue analysis. For example, the mobile application 140 can determine if the image of the tissue sample includes an appropriate number of cells for analysis. The mobile application 140 may use a neural network or other image classification technique to determine if the image of the tissue sample includes a number of cells greater than a threshold value. For example, the mobile application 140 can determine if the image of the tissue sample includes at least five cells, at least one complete cell, at least 20 cells, or some other number. The mobile application 140 can also determine if the image of the tissue sample is of an appropriate type. For example, the mobile application 140 can determine via a neural network or other image classification technique that the image is a SRH image, an image of a hematoxylin and eosin-stained slide, or other type. The mobile application 140 can pre-analyze the image to determine if the image is a valid image that is suitable for analysis. For example, if the image is not a valid image (i.e., is not an image of tissue, is of inadequate resolution, is improperly focused, or otherwise defective), the mobile application 140 can prompt the user to provide new image for analysis.

The mobile application 140 can provide an image of a tissue sample for tissue analysis. In one example, the tissue analysis circuit 145 of the mobile application 140 can be configured to perform a tissue analysis to determine whether the tissue depicted in the image is normal or abnormal. For example, the mobile application 140 may use the tissue analysis circuit 145 stored locally on the mobile device 100 to analyze the image of the tissue sample. In another example, the mobile application 140 can be configured to provide an image of a tissue sample to separate tissue analysis entity, such as a tissue analysis computer system that is located remotely from the mobile device 100. In such examples, the mobile application 140 can transmit the image of the tissue sample to the tissue analysis entity via wireless or wired communication via the network interface circuit 110 or otherwise. In various examples, the mobile application 140 can be configured to provide an image of the tissue sample that meets relevant image standards as specified by the tissue analysis circuit 145 and/or separate tissue analysis entity. For example, the tissue analysis circuit 145 may perform a tissue analysis using images of particular dimensions, file size, color scheme, etc. The mobile application 140 can be configured to determine the relevant image standards by receiving a communication from the tissue analysis circuit 145, the tissue analysis entity.

The mobile application 140 can be configured to provide data regarding the tissue sample to the tissue analysis circuit 145 or other tissue analysis entity (e.g., remotely-located tissue analysis computer system). The mobile application 140 can include the data regarding the tissue sample with the image of the tissue sample as described above or can provide the data regarding the tissue sample in some other manner.

After an image of a tissue sample has been analyzed, any results can be received by the mobile application 140 and can be presented to the user. For example, the mobile application 140 can receive or collect information relating to the tissue sample that is generated or provided by the tissue analysis circuit 145 or another tissue analysis entity. The mobile application 140 can receive an indication from the tissue analysis circuit 145 that a tissue analysis has been successfully generated. In one example, the mobile application 140 can receive a tissue classification result from the tissue analysis circuit 145. The tissue classification result can be an indication that the tissue sample depicted an image of the tissue sample is likely to be abnormal tissue, normal tissue, or some combination thereof, according to one example. The mobile application 140 can present the tissue classification result to the user via a graphical user interface on the display device 120. In another example, the mobile application 140 can present the tissue classification result to a user via the input/output device 105 or via some other means. The tissue classification result can be expressed as an alphanumeric, graphical, or audible notification to the user. In one example, a graphical user interface can be displayed on the display device 120, where the graphical user interface displays the image of the tissue sample and the tissue classification result. The tissue classification result can be displayed as a pop-up notification window over the image of the tissue sample.

After an image of a tissue sample has been analyzed and results have been presented to the user, the mobile application 140 can be configured to prompt the user to take some action. For example, the mobile application 140 can present the user with a selectable option to confirm the result, to store the result, to analyze another image of another tissue sample, or otherwise. The mobile application 140 can store the image of the tissue sample along with a corresponding tissue classification result in a memory of the mobile device 100, such as the memory 135 or some other storage medium (e.g., separate database stored on the mobile device). The mobile application 140 can store the image of the tissue sample and the corresponding tissue classification result according to HIPAA standards and other security protocols. For example, the image and the classification result can be encrypted or accessible only via authenticated users. Users can be authenticated via password, biometric, or other secret knowledge element. In another example, the mobile application 140 can store the image of the tissue sample and the tissue classification in a remotely-located database, such as a database associated with a hospital or surgical group. In such examples, the mobile application 140 can transmit the image of the tissue sample and the tissue classification result via the network interface circuit 110 to at least one remotely-located database. The mobile application 140 can store the image of the tissue sample and the tissue classification result along with the data regarding the tissue sample and any other information relating to the patient, the date and time of a medical procedure, etc. The mobile application 140 can store or transmit (to remotely-located database, user's mobile device, etc.) the image of the tissue sample or the tissue classification result after receiving an indication from the tissue analysis circuit 145 that a tissue classification result has been successfully generated. In other examples, the mobile application 140 can periodically push data or information to a remotely-located database or store data locally, even before the tissue analysis circuit 145 provides an indication that a tissue classification result was successfully generated. In yet other examples, the mobile application 140 can transmit information to a remotely-located database or store data locally only after the tissue analysis circuit 145 provides an indication that the system is in a “ready” state and is ready to analyze another image, for example.

As indicated above, the mobile application 140 can include or be communicably coupled with the tissue analysis circuit 145. The tissue analysis circuit 145 can be structured to differentiate between a normal tissue and an abnormal tissue, according to one example. More specifically, the tissue analysis circuit 145 can be configured to determine whether a particular tissue sample can be characterized as normal tissue or whether it can be characterized as abnormal tissue. In one example, the tissue analysis circuit 145 can determine whether an image of a tissue sample is an image of a normal tissue sample, an abnormal tissue sample, or some combination thereof. The tissue analysis circuit 145 can determine whether an SRH image is an image of normal, healthy tissue or an image of abnormal and/or potentially unhealthy tissue, according to one example.

The tissue analysis circuit 145 can determine whether a tissue sample is normal, abnormal, some combination thereof, or otherwise, by analyzing an image of a tissue sample using artificial intelligence or machine learning techniques. For example, the tissue analysis circuit 145 can include a neural network circuit 150 trained with images of normal and abnormal tissues that can analyze an image of a tissue sample. The neural network circuit 150 can analyze an image of a tissue sample to categorize or classify the image into one or more distinct image classes, such as “normal,” “abnormal,” “tumorous,” “non-tumorous,” “cancerous,” “non-cancerous,” etc. In one example, the neural network circuit 150 can perform an image recognition operation on an image of a tissue sample provided by the mobile application 140 (e.g., an image captured by the optical device 115) and provided to the tissue analysis circuit 145.

The neural network circuit 150 can include a convolutional neural network that includes a plurality of layers each comprising a plurality of neurons to perceive a portion of an image, according to one example. The neural network circuit 150 can be a pre-trained neural network that is further trained using a tissue image dataset. For example, the neural network circuit 150 can be a deeply pre-trained image classifier neural network that has been trained and tested on a large number of images (e.g., over a million images). The neural network circuit 150 can include a pre-trained image set 155 that includes images used to pre-train the neural network circuit 150. The pre-trained image set 155 can be a database stored on the mobile device 100 or can be a remotely-located database stored elsewhere (e.g., a remotely-located computer system). The pre-trained image set 155 can be an ImageNet image set including a relatively large repository of labeled images that can allow a neural network model (e.g., the neural network circuit 150) to learn image classification or to bolster performance in complex computer vision tasks. The neural network circuit 150 can be created or built using a Keras application programming interface, a Pytorch application programming interface, or some other application programming interface. The neural network circuit 150 can include or be based on pre-trained convolutional neural network model, such as a VGG16 convolutional neural network model, an Xception convolutional neural network model, a VGG19 convolutional neural network model, a ResNet convolutional neural network model, a CoreML convolutional neural network model, an Inception convolutional neural network model, or a MobileNet convolutional neural network model. In various embodiments, using a pre-trained neural network can allow the neural network circuit 150 to be trained to recognize whether a tissue is normal or abnormal using a relatively small training dataset, at least as compared to constructing a convolutional neural network anew. In some examples, the training dataset can include a large quantity of images (e.g., 1000 images, 10,000 images, 100,000 images, 500,000 images, or some other amount). The images of the training dataset can be curated images that have been vetted, verified, analyzed, or approved by medical professionals. For example, the images of the training dataset can be images from a database associated with a hospital computing system comprising SRH images from previous patients that have also been analyzed by a pathologist.

The neural network circuit 150 can be trained using a normal tissue image set 160 and an abnormal tissue image set 165. For example, the normal tissue image set 160 can include a plurality of images (e.g., SRH images) of tissue samples that are known to be “normal,” according to pathological analysis or otherwise. In one example, the tissue samples used to create the normal tissue image set 160 can be provided via tissue donations, patients undergoing a surgery, etc. The normal tissue samples can be scanned via a Raman Spectroscopy machine, whereby an SRH image can be generated and displayed on a display device of the Raman Spectroscopy machine. The SRH image can be captured via a camera (e.g., the optical device 115 of the mobile device 100 or otherwise) or can be uploaded or transferred to a computer system for storage. Images used for the normal tissue image set 160 can be processed, reformatted, or resized in order to meet training criteria of the pre-trained neural network model. For example, images can be resized to 224×224 pixels and can be reformatted to an RGB color composition for a Resnet or other convolutional neural network. Images can be resized to 299×299 pixels for a CoreML convolutional neural network model, for example.

The abnormal tissue image set 165 can include a plurality of images (e.g., SRH images, MRI-generated images, CT scan-generated images, or other images) of tissue samples that are known to be “abnormal” according to pathological analysis or otherwise. In one example, the tissue samples used to create the abnormal tissue image set 165 can be tissue samples extracted from a patient during a surgery that have been analyzed (e.g., by a pathologist) to determine that at least a portion of the tissue sample is abnormal. The abnormal tissue samples can be scanned using a Raman Spectroscopy machine to generate an SRH image that is displayed on a display device of the Raman Spectroscopy machine. In one example, the SRH image can be captured via a camera (e.g., the optical device 115 of the mobile device 100 or otherwise) or can be uploaded or transferred to a computer system for storage.

Images used for the abnormal tissue image set 165 can be processed, reformatted, or resized in order to meet training criteria of the pre-trained neural network. For example, images can be resized to 224×224 pixels, 299×299 pixels, or some other size. The images can be reformatted to an RGB color composition or some other color composition. The images can be resized to some other dimension (e.g., 1000×1000 pixels, 3600×3600× pixels). For example, the images used to create the abnormal tissue image set 165 can be whole slide SRH images that can be pre-processed using a Numpy array slicing method to crop and clear the images of non-diagnostic areas. The pre-processing can be completed in Python 3.8, for example. The sliding step for patch creation can be 224 pixels and 299 pixels horizontally and vertically for the Resnet and CoreML models respectively, or other models. The sliding step for patch creation can result in no overlap between patches. For example, the no-overlap method can be used in order to create completely distinct patches for model training, in order to reduce internal model validation bias during the training. All SRH image patches can be manually checked to confirm labels during creation of the abnormal tissue image set 165. Likewise, any regions without visible nuclei can be discarded during creation of the abnormal tissue image set 165.

Using the abnormal tissue image set 165, a deep learning model (e.g., a convolutional neural network model) can be created. For example, a deep learning model can be built using a ResNet50 architecture. The model created with the ResNet50 architecture can be a convolutional neural network with 23 million trainable parameters or some other number of trainable parameters. The ResNet50 architecture can offer superior performance relative to other models in histopathology-based imaging tasks. The model can be altered or fine-tuned with one or more epochs (e.g., 10 epochs, 50 epochs, 100 epochs, or some other number of epochs) utilizing the Adam optimization algorithm. For example, the Adam optimization algorithm can combine Adaptive Gradient Algorithm (AdaGrad) and Root Mean Square Propagation (RMSProp) and can update the network hyperparameters of the model in response to the progress of training. The Adam optimization algorithm can be well-suited for computer vision tasks, for example. The model can be trained using a batch size of 64 images and a learning rate of 3×10⁻⁴. The model can be trained using common data augmentation techniques including rotation and flipping to increase training data. The model's performance can be evaluated using hold-out test dataset with an 80-20 split of the total number of pathology images, for example. The model's performance can be evaluated in some other manner. The model can be built using a Conda miniforge3 (Python 3.8) environment. For example, the Conga miniforge3 environment can be used to build a ResNet-50 model. The model can be build using a 32 GPU core computing device with a 16-core Neural Engine Apple Silicon M1 Max for model building with 64 GB of unified memory.

In other examples, the deep learning model can be built using some other model architecture, such as a CoreML model architecture. For example, the model can be built using CoreML because CoreML can be well-suited to interface with mobile Apple phones or other mobile devices (e.g., an Apple tablet, some other phone, or some other mobile computing device). The abnormal tissue image set 165 can include images sized 299×299 pixels rather than another size (e.g., 224×224 pixels). For example, the abnormal tissue image set 165 can be reacquired by repeating the subdividing of the cleaned pathology images, as described above. The CoreML model can be created a Swift framework using Xcode 12.0, for example. The mobile application 140 can be designed to allow users to take a picture of the SRH screen, implement the deep learning model via the neural network circuit 150, and report a diagnostic certainty in a near-instantaneous manner. The mobile application 140 can be installed on the mobile device 100 having a dual 12MP wide camera or some other high-resolution camera or cameras. The mobile-optimized model (e.g., the CoreML model) can be tested with an 80-20 split from the dataset of pathology images.

The abnormal tissue image set 165 can represent images of a particular type of abnormal tissue. For example, each of the images comprising the abnormal tissue image set 165 can be images of Adenoma pituitary tumor tissue. In such examples, the tissue analysis circuit 145 and the neural network circuit 150 can be configured to determine whether an image of a tissue sample is a normal tissue (e.g., normal pituitary gland tissue) or is an abnormal tissue (e.g., Adenoma pituitary tumor tissue). Accordingly, the tissue analysis circuit 145 and the neural network circuit 150 can analyze an image of a tissue sample to determine whether the tissue depicted in the image is a particular type of normal tissue or a particular type of abnormal tissue. In other examples, the tissue analysis circuit 145 and the neural network can categorize an image of a tissue sample as any number of different types of normal tissue or abnormal tissue.

The neural network circuit 150 can be deployed using the tissue analysis circuit 145 or the mobile application 140 of the mobile device 100 to analyze an image of a tissue sample. In other embodiments, the neural network circuit 150 can be deployed by a web-based application (e.g., web browser) or some other application that can be used.

Upon analyzing an image of a tissue sample, the neural network circuit 150 and/or the tissue analysis circuit 145 can generate a tissue classification result. For example, the tissue classification result can be an indication that the tissue sample depicted in the image is likely to be abnormal tissue or likely to be normal tissue. The tissue classification result can include a probability or confidence interval associated with the tissue classification. For example, the tissue classification result can provide an indication that the tissue depicted in the image is abnormal at a 95% confidence interval, or that there is a 5% margin of error in the tissue classification result. In another example, if the neural network circuit 150 is unable to classify the tissue sample as normal or abnormal, the tissue classification result can state that the result is “indeterminate” or convey a similar message. If the result is indeterminate, the tissue analysis application 330 can prompt the user to provide a new image for analysis or further information regarding the tissue sample, for example. In various examples, the tissue classification result can be provided by the neural network circuit 150 and/or the tissue analysis circuit 145 to the mobile application 140 for presentation to a user. As discussed above, the mobile application 140 can present the tissue classification result to a user via a graphical user interface presented on the display device 120 of the mobile device 100.

The neural network circuit 150 can be configured to perform object detection, semantic segmentation, or instance segmentation when analyzing an image. For example, the neural network circuit 150 can be configured to differentiate between instance of normal tissue and instances of abnormal tissue in a single image of a tissue sample. This can be of particular importance when the image of the tissue sample includes a portion of a tissue sample that is normal and a portion of a tissue sample that is abnormal. In addition to or separately from a tissue classification result, the neural network circuit 150 can provide object detection or segmentation information to the tissue analysis circuit 145 of the mobile application 140. In one example, the mobile application 140 can present the object detection or segmentation information to a use via a graphical user interface presented on the display device 120.

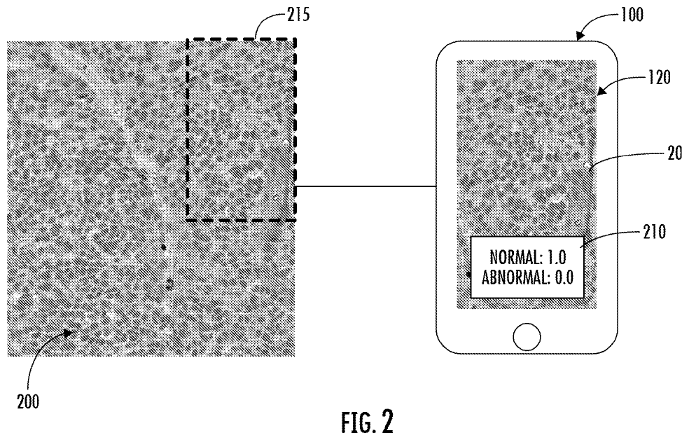

Referring now to FIG. 2, a tissue sample 200 is analyzed by the mobile device 100 to determine if the tissue sample 200 is normal or abnormal. The mobile device 100 can include the tissue analysis circuit 145 and the neural network circuit 150 as discussed above with reference to FIG. 1. Put another way, in the example shown in FIG. 2, the mobile device 100 performs the tissue analysis without relying on a separate tissue analysis entity.

The mobile device 100 can capture an image 205 of the tissue sample 200 using the optical device 115. The image 205 can be an image of a portion 215 of the tissue sample 200, where the portion 215 less than the entire tissue sample 200. In one example, the tissue sample 200 can be a tissue sample displayed on a display device of a Raman Spectroscopy machine. The Raman Spectroscopy machine can scan a physical tissue specimen to generate and display an SRH image of the tissue sample, according to one example. The optical device 115 of the mobile device 100 can capture the image 205 from a display device (e.g., LCD screen) of the Raman Spectroscopy machine for analysis. As discussed above, the image 205 can be captured from within the mobile application 140 of the mobile device 100, where the mobile application 140 can control the optical device 115 to capture the image 205. In another example, the image 205 can be captured via a separate camera application or utility of the mobile device 100 and subsequently uploaded or imported into the mobile application 140 for tissue analysis.

Once the image 205 has been captured and accessed via the mobile application 140, the mobile application 140 can provide the image 205 of the tissue sample 200 to the tissue analysis circuit 145 and/or the neural network circuit 150 of the mobile application 140. The mobile application 140 and/or the tissue analysis circuit 145 can reformat or modify the image 205 of the tissue sample 200 before it is analyzed, such as by reformatting the image, altering the color composition of the image 205, or embedding data regarding the tissue sample (e.g., patient demographics, anatomical location of the tissue sample, etc.). For example, the image can be resized to 224×224 pixels and can be formatted in an RGB color composition before it is provided to the tissue analysis circuit 145 or the neural network circuit 150 for analysis.

The tissue analysis circuit 145 and the neural network circuit 150 can analyze the image 205 to determine if it is normal or abnormal. For example, the neural network circuit 150 can be deeply trained to distinguish between a normal tissue sample and an abnormal tissue sample of a particular type (e.g., normal pituitary gland tissue and Adenoma pituitary tumor tissue). The neural network circuit 150 can be trained using a normal tissue image set 160 and an abnormal tissue image set 165 that are related to the particular type of tissue of the tissue sample 200. Accordingly, the image 205 can be an image of a tissue sample 200 that is either normal, abnormal, or some combination thereof as understood by the neural network circuit 150.

As discussed above, the tissue analysis circuit 145 and the neural network circuit 150 can analyze the image 205 to generate a tissue classification result. The tissue classification result include an indication that the tissue sample 200 (or at least the portion 215 of the tissue sample 200 as represented by the image 205) is normal, abnormal, or some combination thereof. The tissue classification result can also include a confidence interval or some indication of an accuracy of the tissue classification. In another example, the tissue classification result can express the tissue classification in probabilistic terms such that the tissue classification result both indicates what portion of the tissue is classified as normal and what portion is classified as abnormal. In yet another example, the tissue classification result could include a binary result indicated that the tissue sample 200 represented by the image 205 is normal or abnormal.

In another example, the neural network circuit 150 can perform an object detection or image segmentation analysis on the image 205 to determine which portions of the tissue sample 200 depicted in the image 205 are abnormal or normal. For example, the portion 215 of the tissue sample 200 shown in the image 205 can include a first portion comprising normal tissue and a second portion comprising abnormal tissue. The neural network circuit 150 can generate a segmentation result that comprises information regarding any objects detected in the image (e.g., an abnormal tissue portion) and any image segmentation information (e.g., instances of abnormal tissue) that can be provided to the mobile application 140. The neural network circuit 150 can identify both the first portion and the second portion and can provide, to the mobile application 140 information regarding the location of the first portion and the second portion.

The mobile application 140 can receive a tissue classification result and/or a segmentation result from the tissue analysis circuit 145 or the neural network circuit 150. The mobile application 140 can provide the tissue classification result and/or the segmentation result to the user. For example, the mobile application 140 can present a tissue classification widget 210 to the user. The mobile application 140 can provide the tissue classification result and/or the segmentation result to a display device of the Raman Spectroscopy machine. For example, the tissue classification result or segmentation result can be displayed as a heat map or colorized overlay atop the image of the tissue sample on the user device, the display of the Raman Spectroscopy machine, or otherwise. The tissue classification widget 210 can include an alphanumeric depiction of the tissue classification result, according to one example. In another example, the tissue classification widget 210 could be a graphical or audible depiction of the tissue classification result. The tissue classification widget 210 can be displayed over (e.g., on top of) the image 205 of the tissue sample 200. In another example, the tissue classification widget 210 can be a colored or texturized screen that overlays the image 205 on the display device 120, where color or texture of the tissue classification widget 210 conveys information to a user. For example, a translucent color overly could connote object detection or image segmentation information as determined by the neural network circuit 150, where one color can represent portions of the image 205 including abnormal tissue and another color can show portions of the image including normal tissue, according to one example. The portion 215 of the image 205 that has been analyzed can be highlighted (e.g., outlined) on the display device 120 or on the display of the Raman Spectroscopy machine. Portions of the image 205 that have not been analyzed can likewise be highlighted

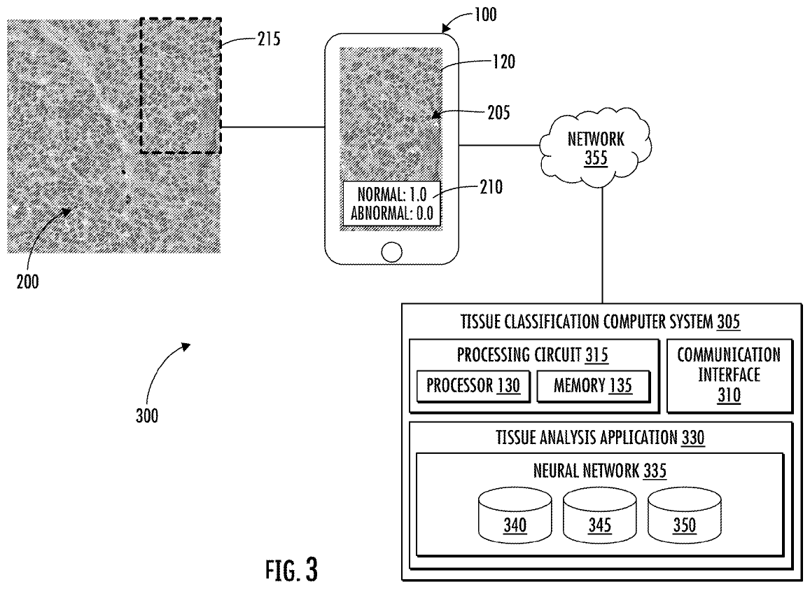

Referring now to FIG. 3, a tissue analysis system 300 is shown. The tissue analysis system 300 can include a tissue analysis computer system 305 that can analyze the tissue sample 200. The tissue analysis computer system 305 can include a communication interface 310, a processing circuit 315, and a tissue analysis application 330. The processing circuit 315 can include a processor 320 and a memory 325. The tissue analysis application 330 can include a neural network 335. In one example, the tissue analysis computer system 305 can analyze the image 205 of a tissue sample 200 to determine if the tissue is normal or abnormal. In another example, the tissue analysis computer system 305 can distinguish one tissue from another tissue. In yet another embodiment, the tissue analysis computer system 305 can be used to determine a characteristic of the tissue sample 200 during a surgical operation (e.g., a tumor removal procedure).

The tissue analysis computer system 305 may be used by a user, such as a surgeon, nurse, pathologist, medical technician, or other medical professional. In one example, the tissue analysis computer system 305 is structured to exchange data over a network 355 via the communication interface 310, execute software applications, access websites, etc. The tissue analysis computer system 305 can be a personal computing device or a desktop computer, according to one example.

The communication interface 310 can include one or more antennas or transceivers and associated communications hardware and logic (e.g., computer code, instructions, etc.). The communication interface 310 is structured to allow the tissue analysis computer system 305 to access and couple/connect to the network 355 to, in turn, exchange information with another device (e.g., the mobile device 100). The communication interface 310 allows the tissue analysis computer system 305 to transmit and receive internet data and telecommunication data with the mobile device 100. Accordingly, the communication interface 310 includes any one or more of a cellular transceiver (e.g., CDMA, GSM, LTE, etc.), a wireless network transceiver (e.g., 802.11X, ZigBee®, WI-FI®, Internet, etc.), and a combination thereof (e.g., both a cellular transceiver). Thus, the communication interface 310 enables connectivity to WAN as well as LAN (e.g., Bluetooth®, NFC, etc. transceivers). Further, in some embodiments, the communication interface 310 includes cryptography capabilities to establish a secure or relatively secure communication session between other systems such as a remotely-located computer system, a second mobile device associated with the user or a second user, a patient's computing device, and/or any third-party computing system. In this regard, information (e.g., confidential patient information, images of tissue, results from tissue analyses, etc.) may be encrypted and transmitted to prevent or substantially prevent a threat of hacking or other security breach.

The processing circuit 315 can include the processor 320 and the memory 325. The processing circuit 315 can be communicably coupled with the tissue analysis application 330 or the neural network 335. For example, the tissue analysis application 330, and/or the neural network 335 can be executed or operated by the processor 320 of the processing circuit 315. The processor 320 can be coupled with the memory 325. The processor 320 can be a general purpose or specific purpose processor, an application specific integrated circuit (ASIC), one or more field programmable gate arrays (FPGAs), a group of processing components, or other suitable processing components. The processor 320 is configured to execute computer code or instructions stored in the memory 325 or received from other computer readable media (e.g., CDROM, network storage, a remote server, etc.).

The memory 325 can include one or more devices (e.g., memory units, memory devices, storage devices, etc.) for storing data and/or computer code for completing and/or facilitating the various processes described in the present disclosure. The memory 325 may include random access memory (RAM), read-only memory (ROM), hard drive storage, temporary storage, non-volatile memory, flash memory, optical memory, or any other suitable memory for storing software objects and/or computer instructions. The memory 325 may include database components, object code components, script components, or any other type of information structure for supporting the various activities and information structures described in the present disclosure. The memory 325 may be communicably connected to the processor 320 via processing circuit 315 and may include computer code for executing (e.g., by the processor 320) one or more of the processes described herein. For example, the memory can include or be communicably coupled with the processor 320 to execute instructions related to the tissue analysis application 330 or the neural network 335. In one example, the memory 325 can include or be communicably coupled with the tissue analysis application 330 or the neural network 335. In one example, the tissue analysis application 330 or the neural network 335 can be stored on a separate memory device located remotely from the tissue analysis computer system 305 that is accessible by the processing circuit 315 via the neural network 335.

The tissue analysis application 330 can be an application or program operated on the tissue analysis computer system 305 that allows a user to perform various operations related to analyzing a tissue sample. For example, the tissue analysis application 330 can be structured to facilitate a user's analysis of an image of a tissue sample (e.g., an SRH image produced by a Raman Spectroscopy machine) to determine whether the tissue depicted in the image is normal tissue or abnormal tissue. For example, the tissue analysis application 330 can facilitate an analysis of the image 205 of the tissue sample 200. In one example, the tissue analysis application 330 can present a tissue classification result to the user, such as by providing a notification via a graphical user interface presented on the display device 120 of the mobile device 100. In various examples, the tissue analysis application 330 can allow a user to provide the image 205 of the tissue sample 200 for analysis and subsequently present the user with a classification of the tissue depicted in the image 205, where the classification can be presented within a short period of time after the image is provided for analysis (e.g., 1-5 seconds, 5-30 seconds, less than 60 seconds, less than three minutes, less than five minutes, etc.).

The tissue analysis application 330 can be configured to receive the image 205 as an input. For example, the tissue analysis application 330 can be communicably coupled with the mobile device 100, where the mobile device 100 can capture the image 205 of the tissue sample 200 using the optical device 115. In one example, the tissue analysis application 330 can obtain, via wireless communication with the mobile device 100, the image 205 of the tissue sample 200 where the image 205 is a previously-captured image of the tissue sample 200. In another example, the tissue analysis application 330 can receive the image 205 of the tissue sample 200 immediately upon capture of the image 205 by the optical device 115 of the mobile device 100 via wired or wireless communication. In yet another example, the tissue analysis application 330 can include a camera function (e.g., camera application) that allows the tissue analysis application 330 to control an optical device (e.g., a webcam) to capture the image 205 of the tissue sample 200. The tissue analysis application 330 can be configured to alter the image 205 of the tissue sample 200 to prepare it for analysis or for some other purpose. For example, the tissue analysis application 330 can reformat the image 205 to ensure that the image 205 has proper dimensions (e.g., 224 pixels by 224 pixels, 1000 pixels by 1000 pixels, 3600 pixels by 3600 pixels, or other dimensions) or has the proper file size (e.g., 1 Mb, less than 1 Mb, less than 5 Mb, less than 20 Mb, greater than 20 Mb, or other size). In another example, the tissue analysis application 330 can ensure that the color of the image 205 is properly calibrated or expressed by converting the image 205 to be compatible with RGB (Red, Green, Blue) color code.

The tissue analysis application 330 can receive a user input regarding the image 205 of the tissue sample 200. For example, the tissue analysis application 330 can receive data (e.g., information, a command, etc.) regarding the tissue sample 200 via a user input provided via the display device 120 or an input/output device 105 of the mobile device 100. In another example, the tissue analysis application 330 can receive information from a user via an input/output device (e.g., a keyboard) coupled with the tissue analysis computer system 305. The data regarding the tissue sample 200 can relate to, for example, an anatomical location of the tissue sample 200 on a patient (e.g., abdominal tissue, pituitary tissue, etc.), demographic information about the patient, or otherwise. In some examples, the data regarding the tissue sample 200 can inform a subsequent tissue analysis by ensuring that a tissue analysis function is properly calibrated or is analyzing the image of the tissue sample with reference to an appropriate sample of known tissue images. In one example, the mobile application 140 can reformat or modify the image 205 of the tissue sample 200 based on the data regarding the tissue sample. For example, the tissue analysis application 330 can resize the image 205 to a particular size that is associated with the particular type of tissue specified by the data regarding the tissue sample 200. The data regarding the tissue sample can be embedded in the image 205 of the tissue sample 200 or otherwise associated with the image 205.

The tissue analysis application 330 can provide the image 205 of the tissue sample 200 for tissue analysis. In one example, the tissue analysis application 330 can be configured to perform a tissue analysis to determine whether the tissue depicted in the image 205 is normal or abnormal. For example, the tissue analysis application 330 may use the neural network 335 stored locally on the tissue analysis computer system 305 to analyze the image 205. In another example, the tissue analysis application 330 can be configured to provide the image 205 to a separate tissue analysis entity, such as a remotely located neural network computer system. In such examples, the tissue analysis application 330 can transmit the image 205 to the separate tissue analysis entity via wireless or wired communication via the communication interface 310 or otherwise. In various examples, the tissue analysis application 330 can be configured to provide the image 205 where the image 205 meets relevant image standards as specified by the neural network 335 and/or separate tissue analysis entity. For example, the neural network 335 can perform a tissue analysis using images of particular dimensions, file size, color scheme, etc. The tissue analysis application 330 can be configured to determine the relevant image standards by receiving a communication from the neural network 335 or the separate tissue analysis entity.

The tissue analysis application 330 can be configured to provide data regarding the tissue sample to the neural network 335 or other tissue analysis entity (e.g., remotely-located neural network computer system). The tissue analysis application 330 can include the data regarding the tissue sample 200 with the image 205 as described above or can provide the data regarding the tissue sample 200 in some other manner.

After the image 205 of a tissue sample has been analyzed, any results can be received by the tissue analysis application 330 and can be presented to the user via the mobile device 100 or via a display device of the tissue analysis computer system 305. For example, the tissue analysis application 330 can receive or collect information relating to the tissue sample 200 that is generated or provided by the neural network 335 or other tissue analysis entity. In one example, the tissue analysis application 330 can receive a tissue classification result from the neural network 335. The tissue classification result can be an indication that the tissue sample 200 depicted in the image 205 is likely to be abnormal tissue, normal tissue, or some combination thereof, according to one example. The tissue analysis application 330 can present the tissue classification result to the user via the mobile device 100, such as by instructing the mobile device 100 to present a graphical user interface on the display device 120. In another example, the tissue analysis application 330 can cause the mobile device 100 to present the tissue classification result to a user via the input/output device 105 of the mobile device 100 or via some other means. The tissue classification result can be expressed as an alphanumeric, graphical, or audible notification to the user. In one example, a graphical user interface can be displayed on the display device 120 of the mobile device 100, where the graphical user interface displays the image of the tissue sample and the tissue classification result. The tissue classification result can be displayed as a pop-up notification window over the image 205 of the tissue sample 200.

After the image 205 of a tissue sample 200 has been analyzed and results have been presented to the user, the tissue analysis application 330 can prompt the user to take some action. For example, the tissue analysis application 330 can cause the mobile device 100 via the mobile application 140 to present the user with a selectable option to confirm the result, to store the result, to analyze another image of another tissue sample, or otherwise. The tissue analysis application 330 can store the image 205 along with a corresponding tissue classification result in a memory of the tissue analysis computer system 305, such as the memory 325 or some other storage medium (e.g., separate database stored on the mobile device). In another example, the tissue analysis application 330 can store the image 205 and the tissue classification in a remotely-located database, such as a database associated with a hospital or surgical group. In such examples, the tissue analysis application 330 can transmit the image of the tissue sample and the tissue classification result via the communication interface 310 to at least one remotely-located database. The tissue analysis application 330 can store the image 205 and the tissue classification result along with the data regarding the tissue sample 200 and any other information relating to the patient, the date and time of a medical procedure, etc.

As indicated above, the tissue analysis application 330 can include or be communicably coupled with the neural network 335. The neural network 335 can be structured to differentiate between a normal tissue and an abnormal tissue, according to one example. More specifically, the neural network can be configured to determine whether a particular tissue sample, such as the tissue sample 200, can be characterized as normal tissue or whether it can be characterized as abnormal tissue. In one example, the neural network 335 can determine whether the image 205 of the tissue sample 200 is an image of a normal tissue sample, an abnormal tissue sample, or some combination thereof. The neural network 335 can determine whether an SRH image, such as the image 205, is an image of normal, healthy tissue or an image of abnormal and/or potentially unhealthy tissue, according to one example.

The neural network 335 can determine whether a tissue sample is normal, abnormal, some combination thereof, or otherwise, by analyzing an image of a tissue sample using artificial intelligence or machine learning techniques. For example, neural network 335 can be a convolutional neural network that is trained with images of normal and abnormal tissues that can analyze an image of a tissue sample, such as the image 205. The neural network 335 can analyze the image 205 to categorize or classify the image 205 into one or more distinct image classes, such as “normal,” “abnormal,” “tumorous,” “non-tumorous,” “cancerous,” “non-cancerous,” etc. In one example, the neural network 335 can perform an image recognition operation on the image 205 (e.g., an image captured by the optical device 115 of the mobile device 100 and transmitted to the tissue analysis computer system 305).

The neural network 335 can include a convolutional neural network that includes a plurality of layers each comprising a plurality of neurons to perceive a portion of an image, according to one example. The neural network 335 can be a pre-trained neural network that is further trained using at least one tissue image dataset. For example, the neural network 335 can be a deeply pre-trained image classifier neural network that has been trained and tested on a large number of images (e.g., over a million images). The neural network 335 can include a pre-trained image set 340 that includes images used to pre-train the neural network 335. The pre-trained image set 340 can be a database stored on tissue analysis computer system 305 or can be a remotely-located database stored elsewhere (e.g., a remotely-located computer system). The pre-trained image set 340 can be an ImageNet image set including a relatively large repository of labeled images that can allow a neural network model (e.g., the neural network circuit 335) to learn image classification or to bolster performance in complex computer vision tasks. The neural network 335 can be created or built using a Keras application programming interface, a Pytorch application programming interface, or some other application programming interface. The neural network 335 can include or be based on pre-trained convolutional neural network model, such as a VGG16 convolutional neural network model, an Xception convolutional neural network model, a VGG19 convolutional neural network model, a ResNet convolutional neural network model, a CoreML convolutional neural network model, an Inception convolutional neural network model, or a MobileNet convolutional neural network model. In various embodiments, using a pre-trained neural network can allow the neural network 335 to be trained to recognize whether a tissue is normal or abnormal using a relatively small training dataset, at least as compared to constructing a convolutional neural network anew.

The neural network 335 can be trained using a normal tissue image set 345 and an abnormal tissue image set 350. For example, the normal tissue image set 345 can include a plurality of images (e.g., SRH images) of tissue samples that are known to be “normal,” according to pathological analysis or otherwise. In one example, the tissue samples used to create the normal tissue image set 345 can be provided via tissue donations, patients undergoing a surgery, etc. The normal tissue samples can be scanned via a Raman Spectroscopy machine, whereby an SRH image can be generated and displayed on a display device of the Raman Spectroscopy machine. The SRH image can be captured via a camera (e.g., the optical device 115 of the mobile device 100 or otherwise) and transferred to the tissue analysis computer system 305, for example. Images used for the normal tissue image set 345 can be processed, reformatted, or resized in order to meet training criteria of the pre-trained neural network model. For example, images can be resized to 224×224 pixels and can be reformatted to an RGB color composition for a Resnet or other convolutional neural network. Images can be resized to 299×299 pixels for a CoreML convolutional neural network model, for example.

The abnormal tissue image set 350 can include a plurality of images (e.g., SRH images) of tissue samples that are known to be “abnormal” according to pathological analysis or otherwise. In one example, the tissue samples used to create the abnormal tissue image set 350 can be tissue samples extracted from a patient during a surgery that have been analyzed (e.g., by a pathologist) to determine that at least a portion of the tissue sample is abnormal. The abnormal tissue samples can be scanned using a Raman Spectroscopy machine to generate an SRH image that is displayed on a display device of the Raman Spectroscopy machine. In one example, the SRH image can be captured via a camera (e.g., the optical device 115 of the mobile device 100 or otherwise) or can be uploaded or transferred to the tissue analysis computer system 305 for storage.

Images used for the abnormal tissue image set 350 can be processed, reformatted, or resized in order to meet training criteria of the pre-trained neural network. For example, images can be resized to 224×224 pixels, 299×299 pixels, or some other size. The images can be reformatted to an RGB color composition or some other color composition. For example, the images used to create the abnormal tissue image set 350 can be whole slide SRH images that can be pre-processed using a Numpy array slicing method to crop and clear the images of non-diagnostic areas. The pre-processing can be completed in Python 3.8, for example. The sliding step for patch creation can be 224 pixels and 299 pixels horizontally and vertically for the Resnet and CoreML models respectively, or other models. The sliding step for patch creation can result in no overlap between patches. For example, the no-overlap method can be used in order to create completely distinct patches for model training, in order to reduce internal model validation bias during the training. All SRH image patches can be manually checked to confirm labels during creation of the abnormal tissue image set 350. Likewise, any regions without visible nuclei can be discarded during creation of the abnormal tissue image set 350.

Using the abnormal tissue image set 350, a deep learning model (e.g., a convolutional neural network model) can be created. For example, a deep learning model can be built using a ResNet-50 architecture. The model created with the Res Net-50 architecture can be a convolutional neural network with 23 million trainable parameters or some other number of trainable parameters. The ResNet50 architecture can offer superior performance relative to other models in histopathology-based imaging tasks. The model can be altered or fine-tuned with one or more epochs (e.g., 10 epochs, 50 epochs, 100 epochs, or some other number of epochs) utilizing the Adam optimization algorithm. For example, the Adam optimization algorithm can combine Adaptive Gradient Algorithm (AdaGrad) and Root Mean Square Propagation (RMSProp) and can update the network hyperparameters of the model in response to the progress of training. The Adam optimization algorithm can be well-suited for computer vision tasks, for example. The model can be trained using a batch size of 64 images and a learning rate of 3×10⁻⁴. The model can be trained using common data augmentation techniques including rotation and flipping to increase training data. The model's performance can be evaluated using hold-out test dataset with an 80-20 split of the total number of pathology images, for example. The model's performance can be evaluated in some other manner. The model can be built using a Conda miniforge3 (Python 3.8) environment. For example, the Conga miniforge3 environment can be used to build a ResNet-50 model. The model can be build using a 32 GPU core computing device with a 16-core Neural Engine Apple Silicon M1 Max for model building with 64 GB of unified memory.

In other examples, the deep learning model can be built using some other model architecture, such as a CoreML model architecture. For example, the model can be built using CoreML because CoreML can be well-suited to interface with mobile Apple phones or other mobile devices (e.g., an Apple tablet, some other phone, or some other mobile computing device). The abnormal tissue image set 350 can include images sized 299×299 pixels rather than another size (e.g., 224×224 pixels). For example, the abnormal tissue image set 350 can be reacquired by repeating the subdividing of the cleaned pathology images, as described above. The CoreML model can be created a Swift framework using Xcode 12.0, for example. The mobile application 140 can be designed to allow users to take a picture of the SRH screen, implement the deep learning model via the neural network circuit 335, and report a diagnostic certainty in a near-instantaneous manner. The tissue analysis application 330 can be installed on the tissue classification computing system 305 configured to receive images from the mobile device 100 over the network 355, where the mobile device 100 can include a camera (e.g., a 12MP wide camera or some other high-resolution camera or cameras). The mobile-optimized model (e.g., the CoreML model) can be tested with an 80-20 split from the dataset of pathology images.

The abnormal tissue image set 350 can represent images of a particular type of abnormal tissue. For example, each of the images comprising the abnormal tissue image set 350 can be images of Adenoma pituitary tumor tissue. In such examples, the neural network 335 can be configured to determine whether an image of a tissue sample is a normal tissue (e.g., normal pituitary gland tissue) or is an abnormal tissue (e.g., Adenoma pituitary tumor tissue). Accordingly, the neural network 335 can analyze the image 205 of the tissue sample 200 to determine whether the tissue depicted in the image 205 is a particular type of normal tissue or a particular type of abnormal tissue. In other examples, the neural network 335 can categorize the image 205 of the tissue sample 200 as any number of different types of normal tissue or abnormal tissue.

The neural network 335 can be deployed using the tissue analysis application 330 to analyze the image 205 of the tissue sample 200. In other embodiments, the neural network 335 can be deployed by a web-based application (e.g., web browser) or some other application that can be used.

Upon analyzing the image 205, the neural network 335 can generate a tissue classification result. For example, the tissue classification result can be an indication that the tissue sample depicted in the image is likely to be abnormal tissue or likely to be normal tissue. The tissue classification result can include a probability or confidence interval associated with the tissue classification. For example, the tissue classification result can provide an indication that the tissue depicted in the image is abnormal at a 95% confidence interval, or that there is a 5% margin of error in the tissue classification result. In another example, if the neural network 335 is unable to classify the tissue sample as normal or abnormal, the tissue classification result can state that the result is “indeterminate” or convey a similar message. If the result is indeterminate, the tissue analysis application 330 can prompt the user to provide a new image for analysis or further information regarding the tissue sample, for example. In various examples, the tissue classification result can be provided by the neural network 335 to the tissue analysis application 330 and further to the mobile device 100 (e.g., via the mobile application 140) for presentation to a user. As discussed above, the tissue analysis application 330 can cause the mobile device 100 to present the tissue classification result to a user via a graphical user interface presented on the display device 120.

The neural network 335 can be configured to perform object detection, semantic segmentation, or instance segmentation when analyzing an image. For example, the neural network 335 can be configured to differentiate between instance of normal tissue and instances of abnormal tissue in a single image of a tissue sample. This can be of particular importance when the image of the tissue sample includes a portion of a tissue sample that is normal and a portion of a tissue sample that is abnormal. In addition to or separately from a tissue classification result, the neural network 335 can provide object detection or segmentation information to the tissue analysis application 330. In one example, the tissue analysis application 330 can cause the mobile device 100 to present the object detection or segmentation information to a user via a graphical user interface presented on the display device 120.

The tissue analysis computer system 305 can thus be used to analyze a tissue sample to determine whether the tissue is normal, abnormal, etc. In this configuration, one or more users operating one or more mobile devices 100 can perform tissue analysis operations by using the tissue analysis application 330 and neural network 335 of the tissue analysis computer system 305 rather than using a neural network circuit 150 of the mobile device as described above with reference to FIG. 1. In this way, a user may only need a mobile application 140 and a tissue analysis circuit 145 that interfaces with the tissue analysis computer system 305 via the network to analyze a tissue sample, thereby reducing a processing burden imposed on the mobile device 100.

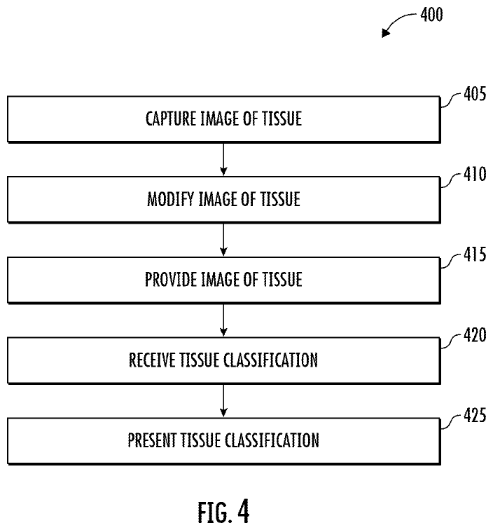

Referring now to FIG. 4, a flow diagram of a method 400 is shown. The method 400 relates to method of analyzing an image of a tissue sample, according to one example. Although the processes 405-425 of the method 400 are discussed below with reference to the mobile device 100, it should be noted that the method 400 can be performed by the mobile device 100, the tissue analysis computer system 305, a combination of the mobile device 100 and the tissue analysis computer system 305, or some other combination of devices.

At process 405, the mobile device 100 can capture an image of a tissue sample. For example, the mobile device 100 can capture an image of a tissue sample to be analyzed via the optical device 115 (e.g., a cell phone camera, separate camera, etc.). The image of the tissue sample can be captured from an SRH image generated by a Raman Spectroscopy machine and displayed on a display device of the Raman Spectroscopy machine, according to one example. In this way, the image captured by the mobile device 100 can be an image of the SRH image rather than an image of the tissue itself. Because the SRH image includes more information (e.g., biological or molecular “fingerprint” information of the tissue) than an ordinary image of the tissue itself, the captured image of the tissue sample may preferably be an image of an SRH image, according to one example. The mobile device 100 can capture the image of the tissue sample via a camera application of the mobile device 100 or can capture the image of the tissue sample from within the mobile application 140.

At process 410, the mobile device 100 can modify the image of the tissue sample. For example, the mobile device 100 can determine that the image of the tissue sample is too large in size (e.g., image dimensions are not appropriate), that the image file size is too large, that the image file has an inappropriate color composition. The image can also be modified by embedding or associating the image with data regarding the tissue sample (e g., demographic information about the patient, anatomical location of the tissue sample, etc.). In various examples, the mobile application 140 or the tissue analysis circuit 145 of the mobile device can be configured to modify the image in accordance with instructions stored in the memory 135 of the mobile device 100. In another example, the tissue analysis circuit 145 or neural network circuit 150 can specify image requirements (e.g., appropriate size, color composition, etc.) and the mobile application 140 can modify the image based on the specified requirements.

At process 415, the mobile device 100 can provide the image of the tissue sample for analysis. For example, the mobile application 140 of the mobile device 100 can provide an image of a tissue sample to the tissue analysis circuit 145 and/or the neural network circuit 150 for image recognition analysis. The neural network circuit 150 can be configured to perform image recognition analyses on the image to determine whether the tissue depicted in the image is a normal tissue, an abnormal tissue, or some combination thereof. In some examples, the tissue analysis circuit 145 and the neural network circuit 150 can be included in the mobile application 140. In such examples, the mobile application can provide instructions to the tissue analysis circuit and/or neural network circuit 150 to analyze the image of the tissue sample. In other examples, the tissue can be analyzed by a separate tissue analysis entity (e.g., the tissue analysis computer system 305 discussed above with reference to FIG. 3). In those circumstances, the mobile device 100 can transmit the image of the tissue sample to the separate tissue analysis entity via the network interface circuit 110.

At process 420, the mobile device 100 can receive a tissue classification. For example, the mobile application 140 can receive a tissue classification result from the tissue analysis circuit 145 and/or the neural network circuit 150 regarding the tissue sample depicted in the image captured at process 405. The tissue classification result can be an indication that the tissue sample includes abnormal tissue, normal tissue, or some combination thereof. The tissue classification can also include object detection or segmentation information related to the tissue sample, including information regarding one or more objects detected in the image or information regarding the location of one or more instances of a certain tissue type (e.g., abnormal tissue) within the image. In examples where the tissue analysis is performed by the tissue analysis circuit 145 and/or the neural network circuit 150 within the mobile application 140, the mobile device 100 may obtain tissue classification information from within the mobile application 140. In other examples, a separate tissue analysis entity (e.g., the tissue analysis computer system 305) can provide the tissue classification to the mobile device 100 via the network interface circuit 110.

At process 425, the mobile device 100 can present the tissue classification to the user. For example, the mobile device 100 can generate a graphical user interface and present the graphical user interface via the display device 120. The graphical user interface can include the image of the tissue sample captured at process 405 as well as a tissue classification widget (e.g., widget 210). The tissue classification widget can include an alphanumeric depiction of the tissue classification result, according to one example. In another example, the tissue classification widget 210 could be a graphical or audible depiction of the tissue classification result. The tissue classification widget 210 can be displayed over (e.g., on top of) the image 205 of the tissue sample 200. In another example, the tissue classification widget 210 can be a colored or texturized screen that overlays the image 205 on the display device 120, where color or texture of the tissue classification widget 210 conveys information to a user. For example, a translucent color overly could connote object detection or image segmentation information as determined by the neural network circuit 150, where one color can represent portions of the image 205 including abnormal tissue and another color can show portions of the image including normal tissue, according to one example.

### B. Systems and Methods Stimulated Raman Histology (SRH) Images to Train a Deep Learning Model for Differentiating Between Different Tissues During Surgery

During surgery—especially tumor surgery—a surgeon usually relies, frequently multiple times in a single surgical intervention, on frozen section pathology answers to make intraoperative critical decisions. Typically, a tissue sample is sent to pathology where, after preparation, it is examined by a pathologist before answers are sent back to the operating room. This process usually takes from 30 to 50 minutes for each specimen sent, depending on the workflow and on the pathologist's availability. Throughout surgery, this wait time increases the duration of the procedure considerably, with longer anesthesia time, higher infection risk, and growing the financial burden. In addition to these disadvantages, in remote centers or in developing countries, access to a specialized pathologist is not always guaranteed. Absence of an onsite pathologist can make this decision process even more complicated.

A relatively new technology uses Raman spectroscopy to generate a biochemical “fingerprint” of a tissue sample by providing simultaneous information on multiple biological molecules and transforming it into an image. This image is visible on a screen in the operating room and generated from unprocessed tissue specimen (without sectioning or staining), in under 3 minutes, enabling rapid histologic evaluation.

### 1. Pituitary Tumors

Pituitary adenomas, accounting for 10-20% of all intracranial neoplasms, are the second most common intracranial neoplasm, with a 17% incidence in autopsy studies of the general population. The majority of pituitary adenomas are benign and non-life threatening. While many tumors are initially observed, surgery is the first-line treatment for tumors demonstrating large size, fast growth, neurovascular compression, or hormone secretion, with the exception of prolactinomas. One of the major challenges in pituitary surgery is differentiating adenoma from normal pituitary gland. Inappropriate gland resection can leave patients with significant hormonal deficiencies. Distinguishing normal gland from abnormal tumor is especially important for functioning adenomas, where any residual disease is associated with higher recurrence rates and has the potential to shorten quantity and quality of life. With gross total resection rates of only 75% for functioning adenomas, surgeons utilize a variety of tools, ranging from intraoperative MRI to 3D image guidance, to improve intraoperative resection rates. Furthermore, a range of pathologies present as a sellar mass, and adenomas can present ectopically within the sphenoid sinus. Hence, recognition of adenoma pathology is a valuable tool for intraoperative decision-making.

During pituitary surgery, there is a frequent need for frozen section to differentiate between tumor (Adenoma) and normal pituitary gland. In fact, that distinction during surgery is critical to allow surgeons to spare the normal gland and to confirm presence of tumor in cases where tumor is not visible to the surgeon or even on the MRI. For that purpose, classically, multiple specimens are sent for frozen section at different times during the procedure. We used the NIO Laser Imaging System, a machine manufactured by Invenio Imaging (and purchased by Memorial Sloan Kettering Cancer Center) to create a large number of images of this particular surgical scenario, which are similar to Hematoxylin & Eosin pathology slides. After generating the images, we created an Artificial Intelligence model to get an automated neural network analysis. This tool was able to differentiate between normal gland and tumor in a few seconds with very high accuracy. The total time, with the Raman image creation, would be reduced to around 3 minutes compared to the traditional frozen section time of 30 to 50 minutes. This would allow more samples to be analyzed intraoperatively in a substantially reduced time. This tool is of particular interest for pituitary surgeons, whereby intraoperative AI-models could deliver fast, real-time information differentiating between normal pituitary gland and adenoma, for guiding surgical decision making and achieving gross total resection of the tumor.

Lightweight convolutional neural networks (CNNs), such as those used in cell phones, may allow more widespread adoption of medical technologies, especially considering that more people have cellphones than potable water. SRH, which uses Raman spectroscopy, a technique that uses light scattering to study the properties of materials, such as their structure and vibrations. It is built on the Raman effect, which was first observed nearly a century ago. SRH provides an opportunity for fusing new, mobile technologies with repurposed, advanced technologies that may become rapidly available in developing nations. Recognizing this potential, we sought to develop a mobile cell-phone app utilizing SRH and prospectively tested our model and app in a clinical trial. We hypothesized that a cellphone-based app could rapidly differentiate between normal pituitary gland and adenoma for rapid, accurate intraoperative pathology.

A phone application can capture images directly from the NIO screen through the phone camera and analyzed them in a few seconds with similar accuracy.

### 2. Other Tumors

The surgical scenario for pituitary tumors can be applied to different other settings. Other neurosurgical and non-neurosurgical tumors can be differentiated using this method. We are working on a similar application for tumor margins in the context of different tumors and organ systems such as various brain tumors, head and neck cancers and others, which can also be analyzed using our tool to provide substantially quicker answers during surgery, with the portability of a tablet or phone app, allowing centers in remote areas or developing countries access to answers in the absence of a specialized pathologist onsite. Other tumors can include, among others, skull base tumors or nasal sinus tumors. Tumors can be located within different anatomical systems and anatomic locations. For example, other neurologic (brain & spine) and ENT tumors, thoracic & mediastinal tumors, prostate/urologic tumors, breast tumors, hepatobiliary/digestive system tumors, skin tumors, bone tumors, gynecologic tumors among others can be analyzed using the systems and methods described herein. Tumors can include benign, precancerous, and cancerous tumors, among others. The systems and methods described herein can also be applied in other contexts, such as in cell morphology and quantification context, or in creating a brain atlas of different cell morphology/density/nuclear/biochemical information

Even though our model was based on the NIO machine, it can be modified and retrained to read traditional Hematoxylin & Eosin slides, or images rendered by other Raman spectroscopy manufacturers (including Bruker Corporation, Photothermal Spectroscopy Corp., Renishaw plc., Horiba Scientific, WITec Wissenschaftliche Instrumente und Technologie GmbH). Ongoing work is focused on developing additional automated neural network analysis models covering different clinical settings, in addition to getting more detailed molecular answers.

### 3. Methods

i. Patient Selection and Study Design

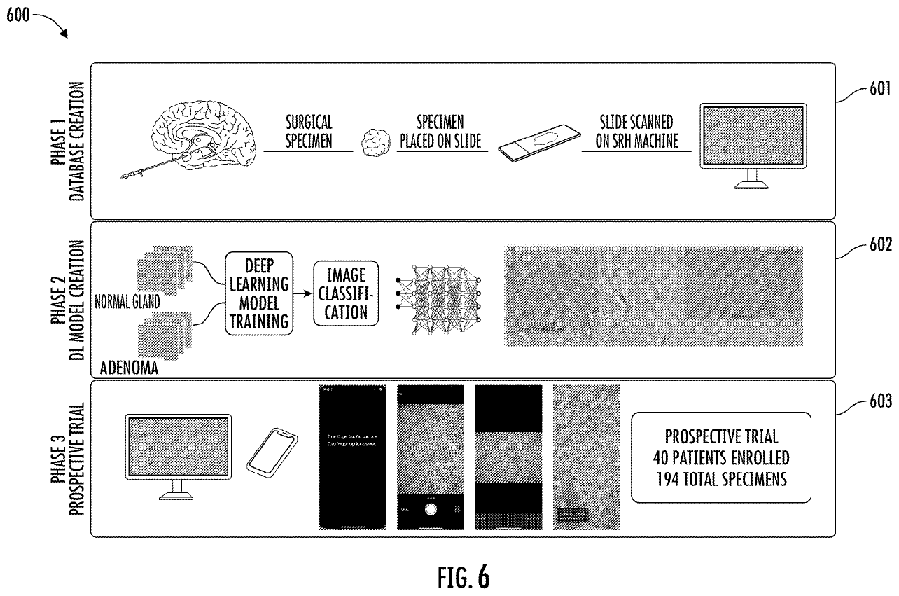

We performed a prospective study developing and deploying a mobile, cellphone-based app using intraoperative SRH to distinguish between normal pituitary gland and pituitary adenoma. For our study, we included consecutive patients from August 2019 through December 2022 who underwent endoscopic transsphenoidal surgery to remove a suspected pituitary adenoma at a single, tertiary referral center. We included all patients, regardless of tumor size, previous operation, or hormone secretory status, to more closely mimic a real-world environment. Our study had three phases: Phase 1 601—a data collection phase; Phase 2 602—building a deep learning-based cellphone app; Phase 3 603—prospective trial evaluating the cellphone app, as depicted in FIG. 6, among others.

All patients provided informed, written consent. We followed the Standards for Reporting Diagnostic Accuracy Studies (STARD) and Transparent Reporting of a Multivariable Prediction Model for Individual Prognosis or Diagnosis (TRIPOD). The research was conducted in accordance with the ethical guidelines on human experimentation from the Helsinki Declaration of 1975. Data can be provided to researchers upon reasonable request.

As depicted in FIG. 6, among others, a flow chart 600 showing three phases—namely Phase 1 601, Phase 2 602, and Phase 3 603 of study are shown. For example, a collection of the images and creation of datasets is shown in Phase 1 601. The deep learning models and the phone application are shown as created in Phase 2 602. Phase 3 603 was a prospective trial to evaluate the phone application performance in a surgical setting.

ii. Image and Dataset Creation

The dataset we used to train our model consists of tumor images and normal tissue images. Tumor images are collected during surgery and normal images from the Last Wish Program (LWP) at Memorial Sloan Kettering. For example, we collected a large dataset of both normal pituitary gland and adenoma pathology images using intraoperative SRH from August 2019 to August 2021. SRH produces images comparable to standard H&E staining through detecting molecular vibrations by scattering monochromatic light. These vibrations are analyzed to determine intracellular components (i.e., lipids, proteins) and reconstructed to form recognizable pathological images that appear similar to H&E stains.

iii. Tumor Images

If deemed safe by the attending neurosurgeon, a small intraoperative sample (e.g., 2 to 20 mm²) was removed and placed on a translucent histology slide without any staining, processing, or sectioning. Most patients had more than one sample, as is standard at our institution. The specimens were small, with a total surface area ranging from 2 to 20 mm² when smeared on the slide. During surgery, a specimen from the tumor is scanned through the SRH machine. An image appears on the screen in the operating room and transferred to our hard drive/PC via USB. Raw images are initially under DICOM format. We performed SRH imaging on each slide using the NIO Laser imaging System by Invenio Imaging Inc. Typically, we scanned an area of 2 mm×2 mm first to have an SRH image displayed quickly, but often expanded image size to cover the whole specimen. The average processing and scanning time for a 2 mm×2 mm scan was 2 minutes. After scanning was complete, we fixed the scanned specimen in formalin and a board-certified neuropathologist provided a formal diagnosis. The neuropathologists' final diagnosis on the scanned specimen served as the “ground-truth” label. We then analyze raw images and filter the parts corresponding to suitable training images, helpful in training the deep learning model to recognize images corresponding to tumor. Images are cropped in a way preserving useful sections and leaving out parts that could be a source of confusion to the model.

To increase the number of normal pituitary gland samples, we collected fresh pituitary glands from the Last Wish Program, a rapid autopsy research program that enables patients at the end of their life to donate their organs for research at Memorial Sloan Kettering Cancer Center. Immediately postmortem, we collected normal pituitary glands from whole body or tissue donations. The specimens were sliced and scanned using the same SRH technique described above.

After scanning was complete, we fixed the scanned specimen in formalin and a board-certified neuropathologist provided a formal diagnosis. The neuropathologists' final diagnosis on the scanned specimen served as the “ground-truth” label. To increase the number of normal pituitary gland samples, we collected fresh pituitary glands from the Last Wish Program, a rapid research autopsy research program that enables patients at the end of their life to donate their organs for research at Memorial Sloan Kettering Cancer Center. Immediately postmortem, we collected normal pituitary glands from whole body or tissue donations. The specimens were sliced and scanned using the same SRH technique described above.

After acquiring whole slide SRH images, pre-processing was performed to develop a large database for model building and to adapt the training image size to the corresponding model. The images were cropped and cleaned of non-diagnostic areas using a Numpy array slicing method in Python 3.8. The database for the ResNet50-architectured model consisted of 224×224 pixel images, while for the CoreML model, the images had a size of 299×299 pixels. The sliding step for patch creation was 224 pixels and 299 pixels horizontally and vertically for the ResNet-50 and CoreML models respectively, resulting in no overlap between patches. The no-overlap method was preferred in order to create completely distinct patches for model training, thereby reducing internal model validation bias during the training. All SRH image patches were then manually checked to confirm labels, and any regions without visible nuclei were discarded

iv. Normal Images

Normal tissue is collected from whole body or tissue donations to the Last Wish Program at MSKCC and immediately scanned, on the SRH machine, to preserve normal living cellular architecture. This normal tissue collection is a significant asset and provides powerful validation.

Raw images are then prepared the same way tumor images are, removing parts that are confusing or not helpful in training the deep learning model to recognize normal tissue.

### V. Model Creation

Using our database of pathology images, we built two different deep learning models. The first model was built using a ResNet-50 architecture, which is a CNN with 23 million trainable parameters. We chose ResNet-50 due to its superior performance in histopathology-based imaging tasks. For our model, we used the version pretrained with ImageNet, a large repository of labeled images that allows CNN models to learn basics of classifying images and improves performance in complex computer vision tasks. We fine-tuned our model with 50 epochs utilizing the Adam optimization algorithm. This algorithm, which combines Adaptive Gradient Algorithm (AdaGrad) and Root Mean Square Propagation (RMSProp), updates the network hyperparameters in response to the progress of training and is well-suited for computer vision tasks. We used a batch size of 64 images and a learning rate of 3×10⁻⁴. We used common data augmentation techniques including rotation and flipping to increase training data. The model's performance was evaluated using hold-out test dataset with an 80-20 split of the total number of pathology images.

We used the Conda miniforge3 (Python 3.8) environment to build and test our ResNet-50 model. We used a 32 GPU core device with a 16-core Neural Engine Apple Silicon M1 Max for model building with 64 GB of unified memory.

Final dataset images are separated into training dataset and testing dataset. The code for creating the model was initially written in Python, using a transfer learning method, which consists of retraining a deeply pretrained image classifier with our own dataset. This method gives deep learning image classification models high accuracy with smaller datasets, because the pretrained model has already been trained and tested on millions of images.

Pretrained models used during this stage can vary. They mostly fall under Keras applications and can be used for feature extraction and prediction. Initially, we used the VGG16 for our first model. But others include Xception, VGG19, ResNet models, Inception models and MobileNet models.

Images are processed and resized to meet training criteria of the model, usually 224×224 RGB images, then, after the model is trained and created, it is tested on a dataset that is completely new and unknown to the model itself. This is the step when the accuracy and performance of each model are tested.

After the model was created, it was initially deployed using Streamlit into a webpage-based app, where the image to be examined could be dragged/uploaded and then analyzed, with a binary classification into Tumor or Normal tissue done in a few seconds.

vi. Phone App and Other Models

We also used alternative development environments like Apple's Xcode, to create the iPhone app where our model was deployed and tested using phone camera capture directly from the image displayed on the SRH screen in the operating room.

In addition to that, we also trained and tested different deep learning models using Apple's Create ML and Google's Teachable Machine, both using MobileNet pretrained models. For example, we built a model using CoreML, which is Apple's machine learning framework that is designed to interface with mobile Apple phones. We used 299×299 images, which is the optimal image size for CoreML. We created our CoreML model in a Swift framework using Xcode 12.0. Our mobile app was designed to allow users to take a picture of the SRH screen, implement the deep learning model, and report a diagnostic certainty in a near-instantaneous manner. We installed our mobile phone app on an iOS 14.1 device (Apple Inc., Cupertino, California, United States) with a dual 12MP wide camera. Similar to the first model, we tested the model performance with an 80-20 split from the dataset of pathology images.

Different interfaces can be used. The webpage-based App is mainly used through a desktop computer, where images are usually under JPG format and size is up to 200 Mb. The phone app, however, uses the phone camera to directly import a photo of the rendered image into the app where the deep learning model is deployed.

Another interesting aspect of our App is that a picture of a small part of the whole slide, containing a few cells can be diagnostic, without analyzing the whole slide architecture. That gives an even better diagnostic tool when the tissue sampled is not sufficient to be examined using conventional pathology techniques, where architecture is a main diagnostic criterion. Having tools to render a diagnosis out of the cellular and molecular aspect of the sample gives an important diagnostic edge, especially when answers determining the next treatment step are not directly available.

We envision broad applications to diverse tumor types, and further modifications to allow utilization by a broad audience of users. Ongoing work will focus on nuclear architecture and coupling the imaging data with transcriptomics.

### 4. Prospective Trial

To test our app, we performed a prospective, blinded trial to evaluate the diagnostic accuracy of consecutive suspected pituitary adenomas from October 2021 through December 2022. The SRH operator was not provided with any information or feedback from a pathologist, thus ensuring that the operator's observations were independent and unbiased. We scanned the specimen using SRH as described above. The operator visually scanned the image for an area with nuclei and took a landscape picture using the mobile app, with the phone at approximately 20 cm from SRH image. Similar to the data collection in Phase 1, the SRH tissue slide was then sent to the neuropathologist for the ground-truth diagnosis.

For our study, we considered a certainty score above 70% as diagnostic. We chose this cutoff based on a survey among board-certified pathologists that defined “consistent with” diagnosis as approximately 70% certainty.²⁵ Lower scores were considered non diagnostic, and we repeated the app evaluation. In all cases, improving the focus and picture quality improved certainty above 70%.

i. Statistical Methods

Using descriptive statistics, we summarized baseline clinical characteristics. To assess model performance discriminating between normal pituitary and abnormal gland, we reported the following metrics: accuracy, sensitivity, specificity, positive predictive value (PPV), negative predictive value (NPV), F1 score, precision, and recall. The F1 score is a commonly used metric in binary classification tasks and is defined as the harmonic mean of precision and recall. Precision is the ratio of true positive predictions to the total number of positive predictions, while recall is the ratio of true positive predictions to the total number of actual positive samples. The F1 score provides a balance between precision and recall, and it is particularly useful when the class distribution is imbalanced. A high F1 score indicates that the model has a good balance of precision and recall, meaning that it can accurately identify both positive and negative samples. Given that our data was clustered, with some patients having multiple pathology specimens per surgery, we developed 95% confidence intervals (CIs) accounting for intra-patient variation using the Taylor series method estimates for variance among clusters.

We displayed the histogram of the certainty for each prediction when classified by ground truth (i.e., normal gland or adenoma) to demonstrate the degree of certainty for each class. The floor for our certainty histograms is 70% as we repeated any sample with a reported certainty below 70%. The certainty score distribution was compared by ground truth using the t-approximation of the Wilcoxon Two-Sample test. We performed all analysis using SAS version 9.4 (The SAS Institute; Cary, NC) and R version 4.2.2 (The R Foundation for Statistical Computing; Vienna, Austria).

### 5. Results

Dataset creation for model training went from August 2019 to September 2021. It included 56 cases where adenoma tissue was sampled and 25 cases where normal pituitary gland was sampled. We supplemented our normal gland dataset with five normal whole pituitary glands collected from fresh autopsies.

After splitting into training and testing datasets, and the preprocessing described in the methods section, a total of 50,603 unique training images were created for the ResNet-architectured model (26,754 adenoma images and 23,849 normal pituitary gland images) with 10,671 testing images (5,781 adenoma images and 4,890 normal pituitary gland images). As for the CoreML model training dataset, it contained 32,051 images (16,694 adenoma images and 15,357 normal pituitary gland images). The testing dataset consisted of 8,013 images (4,174 adenoma images and 3,839 normal pituitary gland images).

After 50 epochs, the ResNet-architectured model rendered an accuracy of 99.3% with a precision of 99.5%, recall of 99.2% and F1 score of 0.99. The CoreML model had a 95.2% accuracy, precision of 93% and recall of 93%, with an F1 score of 0.93.

i. Trial Results (Phase 3)

The prospective trial for the evaluation of the performance of the app in a surgical setting included 40 consecutive patients from October 2021 to December 2022. A total of 194 samples were tested. A neuropathologist evaluated each sample to determine ground truth. The results of the app were compared to ground truth and the following performance measures were obtained: sensitivity was 96.1% (95% CI: 89.9%-99.0%), specificity was 92.7% (95% CI: 74.0%-99.3%), PPV was 98.0% (95% CI: 92.2%-99.8%), and NPV was 86.4% (95% CI: 66.2%-96.8%). Furthermore, the certainty score of the app across all tests was analyzed and the following distribution statistics were obtained: N was 194, minimum was 70.0%, 25th percentile was 86.6%, median was 94.4%, mean was 91.1%, 75th percentile was 98.0%, and maximum was 100.0%.

The certainty score of the App across all tests where the ground truth was normal tissue was analyzed. The following distribution statistics were obtained: N=41, minimum was 70.0%, 25th percentile was 80.8%, median was 91.4%, mean was 88.0%, 75th percentile was 95.7% and maximum was 99.9%.

The certainty score of the phone application across all tests where the ground truth was tumor tissue rendered the following distribution statistics: N=153, minimum was 70.5%, 25th percentile was 88.0%, median was 95.0%, mean was 91.9%, 75th percentile was 98.2% and maximum was 100.0%.

The certainty score was statistically significantly higher when the ground truth was tumor tissue (p=0.0089).

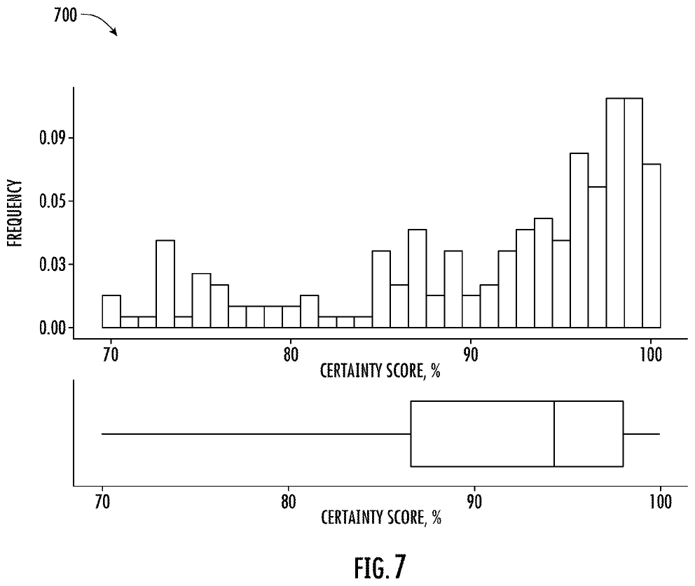

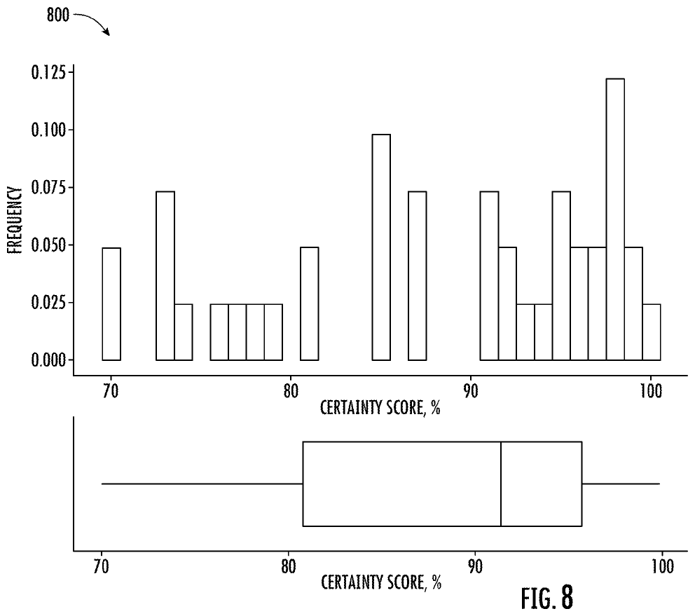

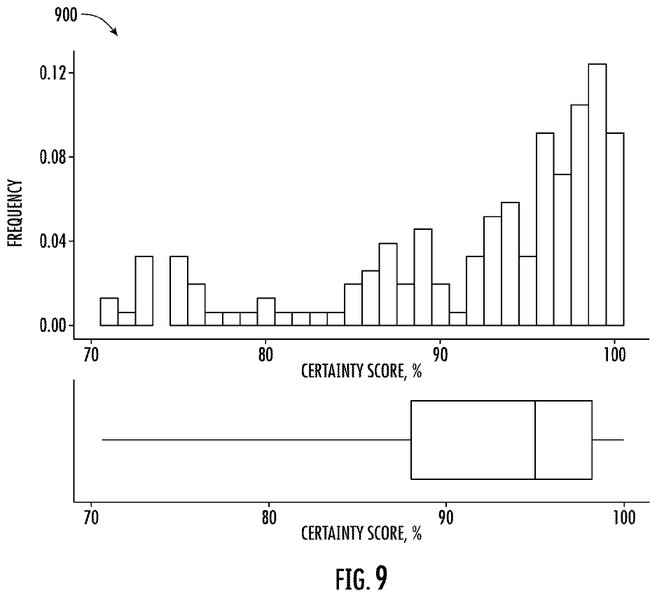

FIGS. 7-9, among others, depict histograms of the certainty of our predictions. For example, FIG. 7 depicts a chart 700 showing a distribution of the mobile application certainty score across all tests. FIG. 8 depicts a chart 800 showing a distribution of the mobile application certainty score across all tests where ground truth was normal tissue. FIG. 9 depicts a chart 900 showing a distribution of the mobile application certainty score across all tests where ground truth was tumor tissue.

### 6. Results

We demonstrated that a deep learning, mobile phone app using simulated Raman histology can successfully differentiate between normal pituitary and adenoma. Pathologists play a vital role in the diagnosis and management of diseases by analyzing tissue samples and identifying abnormalities at the cellular level. However, the global demand for specialized pathologists often exceeds the supply, resulting in a shortage at many centers with neurosurgical expertise. This shortage of pathologists is particularly acute in certain regions, such as sub-Saharan Africa, where the relative number of pathologists is one tenth of most developed nations.

This shortage can lead to delays in diagnosis as samples may need to be sent to distant laboratories for analysis. One possible solution for acute shortages is utilizing AI-based technologies to extend expert physician reach. We propose one solution, utilizing a customized CNN model to achieve intraoperative diagnostic pathology results that are comparable to historical norms. Our cell phone app, prospectively validated in a trial, could be rapidly deployed in resource-limited regions without limited pathological expertise.

Our workflow, which obtained certainty scores within minutes, is a major improvement upon current workflows with H&E staining and interpretation from expert pathologists, often taking 30-50 minutes. Delays in intraoperative pathology reduce surgical effectiveness and can lead to surgeons bypassing the need for intraoperative biopsies which deprives the patients from valuable contribution to surgical decision making. For transsphenoidal pituitary surgery, difficulty differentiating between normal pituitary gland and adenoma can lead to undue damage to normal gland or incomplete resection of viable tumor. Furthermore, extending surgical time while waiting for pathology results, sometimes several times during a single surgery, may increase risks for infections or other intraoperative complications. It is estimated that every 30 minutes of increased surgical time increases the likelihood of a complication by 14%, in addition to increased costs.

Despite obtaining rapid intraoperative pathology results within minutes, our workflow does not sacrifice quality or necessitate expert users. Our F1 score, a common performance marker for binary classification tasks, demonstrates that our model works well. Similarly, we obtained a high degree of confidence, typically exceeding 90%. We achieved this performance with minimal performance loss when using machine learning frameworks designed to work on cell phones (CoreML). Our workflow demonstrates that a high level of accuracy and rapidity can be achieved with minimal technological support, with the potential to extend pathology expertise with easy-to-use technologies.

Ultimately, this system could be applied to different types of histology images and adapted to the local availability of other technologies. A global implementation of this method in adenoma surgery but also other neurosurgical oncology cases and even non neurosurgical tumors would make oncologic surgery safer and offer patients better functional and oncologic outcomes.

### C. Computing and Network Environment

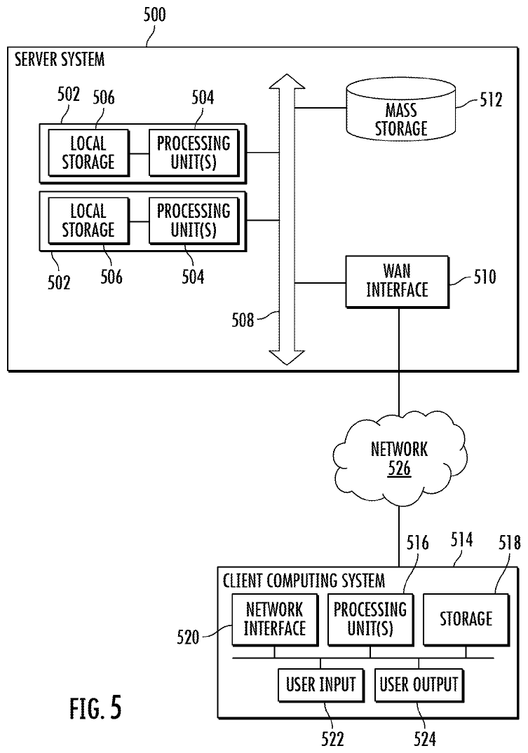

Various operations described herein can be implemented on computer systems. FIG. 5 shows a simplified block diagram of a representative server system 500, client computer system 514, and network 526 usable to implement certain embodiments of the present disclosure. In various embodiments, server system 500 or similar systems can implement services or servers described herein or portions thereof. Client computer system 514 or similar systems can implement clients described herein. The system 305 described herein can be similar to the server system 500. Server system 500 can have a modular design that incorporates a number of modules 502 (e.g., blades in a blade server embodiment); while two modules 502 are shown, any number can be provided. Each module 502 can include processing unit(s) 504 and local storage 506.

Processing unit(s) 504 can include a single processor, which can have one or more cores, or multiple processors. In some embodiments, processing unit(s) 504 can include a general-purpose primary processor as well as one or more special-purpose co-processors such as graphics processors, digital signal processors, or the like. In some embodiments, some or all processing units 504 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 504 can execute instructions stored in local storage 506. Any type of processors in any combination can be included in processing unit(s) 504.

Local storage 506 can include volatile storage media (e.g., DRAM, SRAM, SDRAM, or the like) and/or non-volatile storage media (e.g., magnetic or optical disk, flash memory, or the like). Storage media incorporated in local storage 506 can be fixed, removable or upgradeable as desired. Local storage 506 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 504 need at runtime. The ROM can store static data and instructions that are needed by processing unit(s) 504. The permanent storage device can be a non-volatile read-and-write memory device that can store instructions and data even when module 502 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 506 can store one or more software programs to be executed by processing unit(s) 504, such as an operating system and/or programs implementing various server functions such as functions of the system 305 of FIG. 3 or any other system described herein, or any other server(s) associated with system 305 or any other system described herein.

“Software” refers generally to sequences of instructions that, when executed by processing unit(s) 504 cause server system 500 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 504. Software can be implemented as a single program or a collection of separate programs or program modules that interact as desired. From local storage 506 (or non-local storage described below), processing unit(s) 504 can retrieve program instructions to execute and data to process in order to execute various operations described above.

In some server systems 500, multiple modules 502 can be interconnected via a bus or other interconnect 508, forming a local area network that supports communication between modules 502 and other components of server system 500. Interconnect 508 can be implemented using various technologies including server racks, hubs, routers, etc.

A wide area network (WAN) interface 510 can provide data communication capability between the local area network (interconnect 508) and the network 526, such as the Internet. Technologies can be used, including wired (e.g., Ethernet, IEEE 1302.3 standards) and/or wireless technologies (e.g., Wi-Fi, IEEE 1302.11 standards).

In some embodiments, local storage 506 is intended to provide working memory for processing unit(s) 504, providing fast access to programs and/or data to be processed while reducing traffic on interconnect 508. Storage for larger quantities of data can be provided on the local area network by one or more mass storage subsystems 512 that can be connected to interconnect 508. Mass storage subsystem 512 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 512. In some embodiments, additional data storage resources may be accessible via WAN interface 510 (potentially with increased latency).

Server system 500 can operate in response to requests received via WAN interface 510. For example, one of modules 502 can implement a supervisory function and assign discrete tasks to other modules 502 in response to received requests. Work allocation techniques can be used. As requests are processed, results can be returned to the requester via WAN interface 510. Such operation can generally be automated. Further, in some embodiments, WAN interface 510 can connect multiple server systems 500 to each other, providing scalable systems capable of managing high volumes of activity. Other techniques for managing server systems and server farms (collections of server systems that cooperate) can be used, including dynamic resource allocation and reallocation.

Server system 500 can interact with various user-owned or user-operated devices via a wide-area network such as the Internet. An example of a user-operated device is shown in FIG. 13 as client computing system 514. Client computing system 514 can be implemented, for example, as a consumer device such as a smartphone, other mobile phone, tablet computer, wearable computing device (e.g., smart watch, eyeglasses), desktop computer, laptop computer, and so on.

For example, client computing system 514 can communicate via WAN interface 510. Client computing system 514 can include computer components such as processing unit(s) 516, storage device 518, network interface 520, user input device 522, and user output device 524. Client computing system 514 can be a computing device implemented in a variety of form factors, such as a desktop computer, laptop computer, tablet computer, smartphone, other mobile computing device, wearable computing device, or the like.

Processor 516 and storage device 518 can be similar to processing unit(s) 504 and local storage 506 described above. Suitable devices can be selected based on the demands to be placed on client computing system 514; for example, client computing system 514 can be implemented as a “thin” client with limited processing capability or as a high-powered computing device. Client computing system 514 can be provisioned with program code executable by processing unit(s) 516 to enable various interactions with server system 500.

Network interface 520 can provide a connection to the network 526, such as a wide area network (e.g., the Internet) to which WAN interface 510 of server system 500 is also connected. In various embodiments, network interface 520 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 522 can include any device (or devices) via which a user can provide signals to client computing system 514; client computing system 514 can interpret the signals as indicative of particular user requests or information. In various embodiments, user input device 522 can include any or all of a keyboard, touch pad, touch screen, mouse or other pointing device, scroll wheel, click wheel, dial, button, switch, keypad, microphone, and so on.

User output device 524 can include any device via which client computing system 514 can provide information to a user. For example, user output device 524 can include a display to display images generated by or delivered to client computing system 514. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that functions as both input and output device. In some embodiments, other user output devices 524 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 504 and 516 can provide various functionality for server system 500 and client computing system 514, including any of the functionality described herein as being performed by a server or client, or other functionality.

It will be appreciated that server system 500 and client computing system 514 are illustrative and that variations and modifications are possible. Computer systems used in connection with embodiments of the present disclosure can have other capabilities not specifically described here. Further, while server system 500 and client computing system 514 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method, comprising:
capturing, by an optical reader device of a mobile device, an image of a tissue;
providing, by a mobile application of the mobile device, the image of the tissue to a tissue analysis circuit;
receiving, from the tissue analysis circuit via the mobile device, a tissue classification; and
presenting, via a graphical user interface of the mobile device, a display screen comprising the tissue classification.

2. The method of claim 1, further comprising:
processing, by the mobile application, the image of the tissue prior to providing the image of the tissue to the tissue analysis circuit, wherein processing the image of the tissue includes at least one of resizing the image, reformatting the image, or applying a filter to the image.

3. The method of claim 1, wherein the display screen further comprises the image of the tissue, wherein the tissue classification comprises a pop-up window within the display screen.

4. The method of claim 1, wherein the display screen is presented via the graphical user interface less than one minute after the image of the tissue is provided to the tissue analysis circuit.

5. The method of claim 1, further comprising:
determining, by the mobile application, that the image of the tissue needs to be reformatted according to a tissue analysis specification;
reformatting, by the mobile application prior to providing the image of the tissue to the tissue analysis circuit, the image of the tissue according to the tissue classification in response to the determination that the image of the tissue needs to be reformatted.

6. The method of claim 1, wherein the mobile application comprises the tissue analysis circuit.

7. The method of claim 1, further comprising:
receiving, from the tissue analysis circuit via the mobile application, a request for a second image of the tissue; and
presenting, via the graphical user interface of the mobile device, a second display screen comprising the request for the second image of the tissue.

8. The method of claim 1, wherein the tissue classification is based on an automated neural network analysis performed by a neural network, the automated neural network analysis configured to compare the image of the tissue with a dataset.

9. The method of claim 8, wherein the dataset includes a normal tissue image dataset and an abnormal tissue image dataset, wherein the neural network is a pretrained neural network that is trained to classify the image of the tissue as normal or abnormal.

10. The method of claim 1, wherein the image of the tissue comprises at least a portion of a generated tissue image, the generated tissue image comprising a Stimulated Raman Histology (SRH) image.

11. A mobile device, comprising:
a processing circuit having a processor and a memory, the memory storing instructions that, when executed by the processor, cause the processor to:
receive an image of a tissue;
provide the image of the tissue to a tissue classification circuit;
receive, by the tissue classification circuit based on an automated neural network analysis, a classification of the image of the tissue; and
present, via a display device, a display screen comprising the classification of the image of the tissue, the classification comprising an indication that the tissue is normal or abnormal.

12. The mobile device of claim 11, comprising:
an optical reader configured to capture an image, wherein the image of the tissue is captured by the optical reader from a generated Stimulated Raman Histology image displayed on an imaging device.

13. The mobile device of claim 11, wherein the tissue classification circuit comprises a neural network configured to perform the automated neural network analysis, the neural network trained to classify the image of the tissue as normal or abnormal using a normal tissue image dataset and an abnormal tissue dataset.

14. The mobile device of claim 11, wherein the instructions further cause the processor to:
process, by the mobile device, the image of the tissue prior to providing the image of the tissue to the tissue classification circuit, wherein processing the image of the tissue includes at least one of resizing the image, reformatting the image, or applying a filter to the image.

15. The mobile device of claim 11, wherein the instructions further cause the processor to:
determine, by the mobile device, that the image of the tissue needs to be reformatted according to a tissue analysis specification;
reformat, by the mobile device prior to providing the image of the tissue to the tissue classification circuit, the image of the tissue according to the tissue classification in response to the determination that the image of the tissue needs to be reformatted.

16. The mobile device of claim 11, wherein the display screen is presented via the display device less than one minute after the image of the tissue is provided to the tissue classification circuit.

17. A system, comprising:
an imaging device comprising a display device, the imaging device configured to generate a Stimulated Raman Histology (SRH) image of a tissue and display the SRH image on the display device; and
a tissue classification computer system coupled to the imaging device, the tissue classification computer system comprising a neural network trained with a normal tissue image dataset and an abnormal tissue image dataset, wherein the tissue classification computer system is configured to:
receive the SRH image of the tissue;
perform an automated neural network analysis to classify at least a portion of the SRH image of the tissue as normal or abnormal; and
provide an indication of a classification of the SRH image of the tissue as normal or abnormal.

18. The system of claim 17, wherein the neural network is a pre-trained neural network that is trained using the normal tissue image dataset and the abnormal tissue image dataset to classify an image of tissue as normal or abnormal.

19. The system of claim 17, wherein the tissue classification computer system is further configured to:
select the portion of the SRH image of the tissue, wherein the automated neural network analysis is performed on the selected portion of the SRH image of the tissue.

20. The system of claim 17, wherein the indication of the classification of the SRH image of the tissue is provided, by the tissue classification computer system, to the display device of the imaging device.
